(19) United States
(12) Patent Application Publication
(10) Pub. No.: US 2009/0234131 A1
(43) Pub. Date: Sep. 17, 2009

(54) INFRARED DYE FOR SILVER HALIDE-BASED PHOTOGRAPHIC ELEMENTS

(75) Inventors: Paul L. Zengerle, Rochester, NY (US); John W. Harder, Rochester, NY (US); Steven P. Szatynski, Rochester, NY (US); Stephen P. Singer, Spencerport, NY (US)

Correspondence Address: Raymond L. Owens, Patent Legal Staff;Eastman Kodak Company, 343 State Street, Rochester, NY, 14650-2201, US

(73) Assignee: Eastman Kodak Company, Rochester, NY (US)

(21) Appl. No.: 12/416,212

(22) Filed: Apr. 1, 2009

Related U.S. Application Data
(62) Division of application No. 12/047,342, filed on Mar. 13, 2008.

(51) Int. Cl. C07D 277/62

(52) U.S. Cl. 548/156

(57) ABSTRACT

A non-infrared sensitized low silver photographic element with a non-light sensitive layer, preferably an antihalation layer, containing a diphenylaminocyclopentene heptacyanine benzothiazolium infrared dye where the benzothiazolium groups have electron-withdrawing substituents on the phenyl ring and solubilzed alkyl groups on the nitrogen. The infrared dye can be present as a liquid-crystalline dispersion. This class of infrared dyes form J-aggregrated species in a liquid-crystalline or a solid particle dispersion with high IR density and low visible absorbance.

### FIELD OF THE INVENTION

This invention relates to infrared dyes and their use to increase the infrared density of silver halide-based photographic elements before processing.

### BACKGROUND OF THE INVENTION

It is very desirable to reduce the raw material costs of producing silver halide-based photographic elements by reducing the total amount of silver used. However, there is a limit to the amount the imaging silver (silver halide) can be reduced since the photographic element still must meet aim specifications of speed, contrast and maximum density. Imaging silver is defined as the light sensitive silver halide present in imaging layers and does not include any metallic non-light sensitive silver present such as Lippmann silver or colloidal silver. Many photographic elements, particularly color negative films, use metallic silver in its black colloidal form (often referred to as grey silver) in an antihalation layer to prevent light scatter. The amount of colloidal silver used is generally determined by the amount of antihalation protection required.

Automated processing systems and machines for photographic elements depend on sensors to detect when an element is present at various stages of the process of photographic development. Almost all such systems and machines use an infrared (IR) emitting device (such an IR laser) and an IR detector as the sensing unit. IR emitters generally have relatively narrow IR emission (920-980 nm, with maximum emission about 950 nm. IR detectors are generally sensitive over a broad band (typically 600-1100 nm, with maximum sensitivity about 800 nm. As a result, almost all automated processing systems use IR sensing units that are sensitive in the range of 900-1000 nm, with the maximum sensitivity being around 920-980 nm.

IR sensing units are suitable for this use since photographic elements have high IR density due to the fact that both silver halide and metallic silver inherently absorb IR light. For a machine standpoint, IR sensing units are desirable since they are cheap and reliable in operation. It should be noted that without special sensitizing, IR radiation does not cause development of silver halide.

However, the IR density of the photographic element to be detected must be high enough above the background noise to always be detected without false detection. Hence the amount of IR density to be detected is relatively high, at least 1.2 and more typically, greater than about 1.6.

Over the years, many improvements have been made in the efficiency of the imaging process so that much less silver halide is required. However, lower silver halide levels (with the resulting thinner film packages) has an offsetting effect on the amount of metallic silver required for antihalation since more light passes through the package and is available for scatter even though a thinner package decreases the amount of halation. Higher levels of colloidal metallic silver are often undesirable since it leads to increased fog and Dmin in neighboring imaging layers as well as affecting their development.

In any case, the utilization of silver halide for imaging in photographic elements has become so efficient that although such low silver films meet aim performance specifications, they are insufficient in IR density for the IR detectors in automated processing systems. Since metallic silver has inherently higher IR absorbance than silver halide, the amount of metallic silver can be increased to meet the overall IR density requirements as well as increased halation protection but this can also lead to higher fog levels and other problems.

U.S. Pat. No. 6,210,871 proposes the use of a preformed IR dye in a silver halide color photographic material with low silver to increase the IR density. However, the type of IR dyes used, as typified by Cmpd-32 in Column 13-14 and similar in structure to IR-140 below, have low extinction in the IR. U.S. Pat. No. 5,714,307 and U.S. Pat. No. 5,853,969 disclose the use of the same types of preformed IR dyes in a silver halide photographic material as a dispersion of solid particles.

In addition to Cmpd-32 above, other diphenylaminocyclopentene heptacyanine benzothiazolium IR dyes are known. Another specific example of this type of IR dye based on a 2-[2-[3-[(2(3H)-benzothiazolylidene)ethylidene]-2-(diphenylamino)-1-cyclopenten-1-yl]ethenyl]benzothiazolium salt is IR-140 (CAS RN 53655-17-7) which has the following structure:

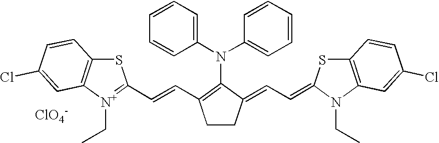

References that describe that the use of this class of dye for sensitization of silver halide to IR radiation include U.S. Pat. No. 5,057,406; JP06105340; U.S. Pat. No. 4,536,473; EP88595; and U.S. Pat. No. 3,671,648. Also included are JP10239834; W. Freyer et al, Zeitschrift fuer Chemie (1989), 29(3), 105-6; and U.S. Pat. No. 3,671,648 which specifically describe compounds where the benzothiazolium groups are substituted with chloro groups.

References that describe that the use of this class of dye for use in an IR laser include B. Pierce et al, IEEE Journal of Quantum Electronics (1982), QE18(7), 1164-70; JP Fouassier et al, Optics Communications (1977), 23(3), 393-7; J P Webb et al, IEEE Journal of Quantum Electronics (1975), QE11(3), 114-19; and U.S. Pat. No. 3,774,122.

U.S. Pat. No. 5,219,707 describes the use of this class of dye for use as an IR sensitizer in an optical disk film.

References that describe that the use of this class of dye for use as an IR sensitizer for electrophotography include JP04042428 and U.S. Pat. No. 4,418,135. In particular, CA1247915 which describes compounds where the benzothiazolium groups are substituted with trifluoromethyl or nitro groups for this use.

U.S. Pat. No. 6,527,977 describes the preparation and use of dyes and other materials as liquid-crystalline dispersions. U.S. Pat. No. 5,718,388, U.S. Pat. No. 5,500,331 and U.S. Pat. No. 5,478,705 all describe the preparation and use of dyes and other materials as solid particle dispersions.

Despite a number of attempts to provide silver halide photographic materials with low silver and sufficient IR density by the addition of a preformed IR dye, there still remains a need for preformed IR dyes with improved properties. In a particular embodiment, the problem remains to provide a low cost silver halide photographic element having low silver exhibiting low fog but with sufficient IR density that may be detected by automated processing systems.

### SUMMARY OF THE INVENTION

A non-infrared sensitized photographic element comprising a support; at least one light-sensitive silver halide emulsion layer and a non-light sensitive layer containing an infrared dye according to Formula (I):

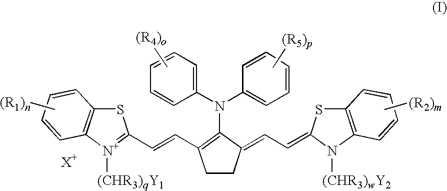

wherein:

R₁ and R₂ each independently represent substituents chosen such that the sum of the Hammett σₚₐᵣₐ and σₘₑₜₐ parameters of R₁ and R₂ in total is greater than 0.45;

R₃ is hydrogen or an alkyl group;

R₄ and R₅ each independently represent substituents;

n, m, o and p each independently represents an integer from 0-4 and at least one of n or m is at least 1;

q and w each independently represents an integer from 1-4;

Y₁ and Y₂ each independently represents an anionic carboxylic acid group, a sulfonic acid group or a N-sulfonylcarbamoyl group; and

X is a counterion chosen so that the net charge of the entire molecule is zero.

In one embodiment, the non-light sensitive layer is an antihalation layer. In another embodiment, the infrared dye is present as a liquid-crystalline dispersion. Also provided are a class of infrared dyes that form J-aggregated species in a liquid-crystalline dispersion with high IR density and low visible absorbance.

This invention provides IR dyes with high IR and low visible absorbance that can be used to provide silver halide photographic elements with low silver and high IR density with low manufacturing cost and low Dmin.

### BRIEF DESCRIPTION OF THE DRAWINGS

FIG. 1 shows a plot of density versus wavelength for inventive and comparative infrared dyes in different dispersion formulations.

### DETAILED DESCRIPTION OF THE INVENTION

The invention is generally as described above.

Typically, the silver halide photographic element useful in one aspect of the present invention is a color element which comprises a support, bearing an antihalation layer comprising colloidal metallic silver, a cyan dye image-forming unit comprised of at least one red-sensitive silver halide emulsion layer having associated therewith at least one cyan dye-forming coupler, a magenta dye image-forming unit comprising at least one green-sensitive silver halide emulsion layer having associated therewith at least one magenta dye-forming coupler, and a yellow dye image-forming unit comprising at least one blue-sensitive silver halide emulsion layer having associated therewith at least one yellow dye-forming coupler. In another embodiment, it is also possible that the separate color forming layers are collapsed into one or more layers so that the element produces only neutral images. Any such imaging elements may be processed via thermal means only or can be processed using phenylenediamine based developers. It is preferred that the color silver halide elements are negative working silver halide elements. It is also preferred that the silver halide photographic elements are capture or origination elements such as a color negative film or a motion picture origination film.

An infrared (IR) dye is an organic dye that absorbs light in the range of 780-1100 nm. In order to minimize the amount of IR dye required to increase the IR density of the film element to the desired level, it is most desirable to have the maximum absorbance of the dye match the maximum sensitivity of the IR sensing unit, namely around 920-980 nm. However, for other uses, the IR absorbance can be over a broader region, namely 900-1010 nm.

The IR dye of the invention is preformed; that is, it is present prior to any exposure and processing and is not formed in-situ during processing via reaction with Dox. The IR dye of the invention is used in a non-light sensitive layer, does not sensitize silver halide to IR light and is present in a non-imagewise and uniform fashion across the element. Although the IR dye may be partly or wholly destroyed in or removed from the element during any of the processing steps, it is desirable that the bulk of IR dye or its decomposition fragments remain in the film after processing to prevent seasoning of the processing solutions. It is also highly desirable that the IR dye has little or no absorbance in the visible region since if left in the film after processing, it would raise the Dmin of the film element, or color the processing solutions if seasoned into them.

The IR dye of the invention is a diphenylaminocyclopentene heptacyanine benzothiazolium dye according to Formula (I):

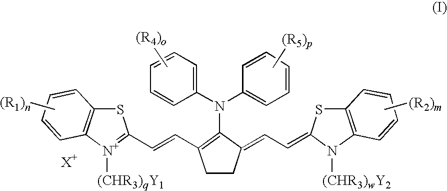

wherein:

R₁ and R₂ each independently represent substituents chosen such that the sum of the Hammett σₚₐᵣₐ and σₘₑₜₐ parameters of R₁ and R₂ is greater than 0.45;

R₃ is hydrogen or an alkyl group;

R₄ and R₅ each independently represent substituents;

n, m, o and p each independently represents an integer from 0-4 and at least one of n or m is at least 1;

q and w each independently represents an integer from 1-4;

Y₁ and Y₂ each independently represents an anionic carboxylic acid group, a sulfonic acid group or a N-sulfonylcarbamoyl group; and

X is a counterion chosen so that the net charge of the entire molecule is zero.

In Formula (I), suitable groups for R₁ and R₂ include halide, an alkyl group, an aryl group, an alkoxy group, an aryloxy group, an oxycarbonyl group (—OCOR), an ester of a carboxylic acid (—CO₂R), a carbonamide group (—NR—COR), a carbamoyl group (—CONR₍₂₎), a thioether group, a sulfoxide group, a sulfone group, a cyano group, a heterocyclic group or a nitro group. Two adjacent R₁ or R₂ cannot be joined together to form an annulated aromatic ring. The group R in the above can be hydrogen, a substituted or unsubstituted alkyl group including methyl, ethyl, n-butyl or t-butyl or a substituted or unsubstituted aryl group such as phenyl, naphthyl or p-chlorophenyl.

Suitable groups for R₃ include an alkyl group of 1-4 carbon atoms which may be branched and which can be further substituted.

Suitable groups for R₄ and R₅ include halide, an alkyl group, an aryl group, an alkoxy group, an aryloxy group, an oxycarbonyl group (—OCOR), an ester of a carboxylic acid (—CO₂R), a carbonamide group (—NR—COR), a carbamoyl group (—CONR₂), a thioether group, a sulfoxide group, a sulfone group, a cyano group, a heterocyclic group, an amino group or a nitro group. Two adjacent R₄ or R₅ can be joined together to form an annulated aromatic ring. The group R in the above can be hydrogen, a substituted or unsubstituted alkyl group including methyl, ethyl, n-butyl or t-butyl or a substituted or unsubstituted aryl group such as phenyl, naphthyl or p-chlorophenyl.

Y₁ and Y₂ are water-solubilizing groups and must be ionizable. A N-sulfonylcarbamoyl group has the following structure:

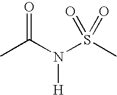

where the hydrogen on the nitrogen can be ionized.

Suitable groups for X include alkali metal cations including Li⁺, Na⁺ and K⁺, alkaline earth metal cations including Ca⁺² and Mg⁺², ammonium or alkyl or aryl ammoniums salts including NH₄⁺, Me₃NH⁺, Et₃NH⁺ or PhNH₃⁺, or quaternary salts such as (Me)₄N⁺.

A more preferred formula for useful IR dyes is according to Formula (II) using the same definitions as in Formula (I):

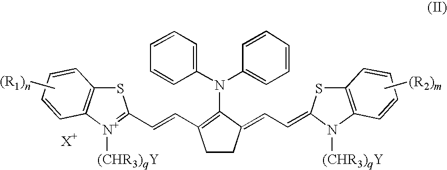

wherein:

R₁ represent substituent(s) chosen so that the sum total of the Hammett σₚₐᵣₐ and σₘₑₜₐ parameters of all R₁ are greater than 0.23;

R₂ represent substituent(s) chosen so that the sum total of the Hammett σₚₐᵣₐ and σₘₑₜₐ parameters of all R₂ are greater than 0.23; n and m each independently is 1 or 2;

R₃ is hydrogen or an alkyl group;

q represents an integer from 1-4;

Y is a carboxylic acid group, a sulfonic acid group or N-sulfonylcarbamoyl group; and

X is a counterion chosen so that the net charge of the entire molecule is zero.

The most preferred structure for the IR dye is according to Formula (III) using the same definitions as in Formula (I):

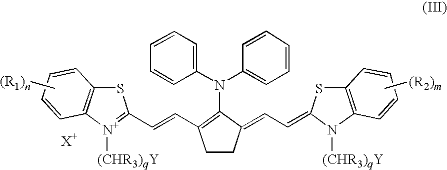

wherein:

R₁ and R₂ are the same and each is a halide, an ester of a carboxylic acid (—CO₂R), a carbamoyl group (—CONR), a sulfoxide, group, a sulfone group, a cyano group, a heterocyclic group or a nitro group and chosen such that the sum of the Hammett σₚₐᵣₐ and σₘₑₜₐ parameters of R₁ and R₂ is greater than 0.75;

n and m each independently represents an integer 1 or 2;

R₃ is hydrogen or an alkyl group;

q is 3 or 4; and

Y is a sulfonic acid group; and

X is a counterion chosen so that the net charge of the entire molecule is zero.

The definition and determination of Hammett σ parameters for various substituents are well known in the chemical arts. Hammett o parameters come in two forms, σₘₑₜₐ and σₚₐᵣₐ, depending on the position of the substituent in question relative to another substituent on the same phenyl ring. In this case, σₘₑₜₐ is used when the substituent is located meta to the sulfur of the benzothiazolium group and σₚₐᵣₐ when it is located para to the sulfur. The sum of all Hammett σ parameters refers to the total of the appropriate σₘₑₜₐ and σₚₐᵣₐ values of all substituents on the phenyl ring. The following table lists some typical values for Hammett a parameters and is taken from A. Gordon and R. Ford, The Chemist's Companion: A Handbook of Practical Data, Techniques and References, Wiley-Interscience, John Wiley& Sons, NY, 1972, pp 145-147. Note that in dispersion, highly acidic groups (pKₐ<6) such as carboxylic and sulfonic acids will be in their ionized forms and the Hammett σ parameters for the ionized form should be used.

| Substituent | σₘₑₜₐ | σₚₐᵣₐ |
|---|---|---|
| —CH₃ | −0.069 | −0.170 |
| —C₄H₉-t | −0.100 | −0.197 |
| —C₆H₅ | 0.060 | −0.010 |
| —CN | 0.560 | 0.660 |
| —COCH₃ | 0.376 | 0.502 |
| —CONH₂ | 0.280 | 0.360 |
| —CO₂⁻ | −0.100 | 0.000 |
| —CO₂CH₃ | 0.320 | 0.390 |
| —CO₂C₂H₅ | 0.370 | 0.450 |
| —CF₃ | 0.430 | 0.540 |
| —NHCOCH₃ | 0.210 | 0.000 |
| —NO₂ | 0.710 | 0.778 |
| —OCH₃ | 0.115 | −0.268 |
| —OC₆H₅ | 0.252 | −0.320 |
| —F | 0.337 | 0.062 |
| —Cl | 0.373 | 0.227 |
| —Br | 0.391 | 0.232 |
| —SOCH₃ | 0.520 | 0.490 |
| —SO₂CH₃ | 0.560 | 0.680 |
| —SO₃⁻ | 0.050 | 0.090 |

The IR dyes of Formula (I) are particularly desirable since they are excellent for forming J-aggregates in non-polar conditions, such as in the solid or liquid-crystalline states. The J-aggregated forms of these IR dyes have significantly narrower and deeper (higher wavelength) absorbance bandwidth than non-aggregated IR dyes, thus lowering the amount of unwanted visible absorbance. In addition, the narrow bandwidth of the J-aggregate effectively increases the extinction coefficient greatly at the wavelength of maximum absorbance (Lₘₐₓ). In this regard, it may be necessary to ‘tune’ the maximum absorbance of the J-aggregate to match the maximum sensitivity of the IR sensor by either substituent changes or by modifying the dispersion environment. In some situations, it might be necessary to use two different dyes in combination in order to cover a wider range than either alone. For a discussion of J-aggregates, see ‘The Theory of the Photographic Process, 4^{th} Edition’, T. H. James, Ed., Macmillan Publishing Co. NY, 1977, pp 218-222.

The IR dye of the invention is located in a non-light sensitive layer in the imaging element. The layer with the IR dye should not contain any silver halide that is sensitized to light or contributes significantly to the image formed after processing. The layer may be located anywhere other than an imaging layer within the photographic element. For example, the IR dye may be located in a protective overcoat on top of imaging layers (and furthest from the support), an interlayer between an imaging layer and the protective overcoat, in an interlayer between any two imaging layers, an interlayer between an imaging layer and the antihalation layer, the antihalation layer, an interlayer between the antihalation layer and the support or in a layer on the support opposite to the imaging layers. Any of these layers may contain other components useful in those layers such as other dyes, scavengers and the like.

The most preferred location is the antihalation layer which additionally contains either colored dyes or more preferably, black colloidal metallic silver (“gray silver”) to provide halation protection. In addition to the materials that provide halation protection, the antihalation layer also commonly contains scavengers for oxidized developers, preformed dyes to adjust Dmin and passivating materials for the metallic silver if present.

The IR dye of the invention is not significantly water-soluble and should not diffuse into other layers upon long-term storage before processing nor diffuse out of the element intact during processing. They are typically used as dispersion; that is, a finely divided state suspended in a medium. Suitable dispersions are either as a solid particle dispersion (see U.S. Pat. No. 5,718,388, U.S. Pat. No. 5,500,33 1 and U.S. Pat. No. 5,478,705) or as a liquid-crystalline dispersion (see U.S. Pat. No. 6,527,977 for a discussion of liquid-crystalline dispersions and their preparation). In some cases, the dye, dissolved in an alcohol or other water-miscible organic solvent, can be added directly to the other components of the layer, where the dye will form a micellar dispersion.

In order to be detected by an IR sensor in an automated processing system, an imaging element should have an IR density at 950 nm, of at least 1.2, more desirably at least 1.4 or most preferably at least 1.6. The two main contributors to the overall IR density of a silver halide based photographic element are the silver halide imaging emulsions and metallic silver. Even though silver halide is present at higher levels, the metallic silver has a large effect of the total IR density since it has a larger inherent IR absorbance. The amount of inherent IR absorbance of these species can vary with their physical characteristics as well as surface treatments. Since the amount of IR density will depend on both the amount and the characteristics of silver present, the exact amount of additional IR density required or the minimum amount of total silver present to have sufficient IR density cannot be predicted except on a case-by-case basis. The exact amount of IR dye required will depend on its effective extinction coefficient as judged by the IR sensor. Once an optimum level of silver halide and metallic silver is determined to be sufficient to meet the aim performance specifications, any deficiency can be made up by the addition of a suitable amount of IR dye. Generally, for typical color negative silver halide photographic films without any additional IR dye, films with imaging silver levels above about 3.0 g/m² are expected to have sufficient IR density but films with imaging silver levels of about 2.8 g/m² or less may have insufficient IR. These film elements would need to include additional colloidal silver for the sole purpose of adding IR density. The additional colloidal silver would have the undesired side effect of causing fog in adjacent layers.

As discussed above, the infrared dyes according to this invention have narrower bandwidths with high extinction coefficients and low visible absorbance because of their ability to form J-aggregates when dispersed either in a solid or liquid-crystalline form. This is believed to be due to the combination of electron-withdrawing substituents on the benzothiazolium groups and an ionized acid group on the alkyl nitrogen substituent. This may allow their use in other applications where IR dyes with high extinction and low visible absorbance are desirable. Suitable infrared dyes have a maximum absorption between 900 nm and 1010 nm, preferably between 920-980 nm, when dispersed as in either a solid or a liquid-crystalline state and have a structure according to Formula (III):

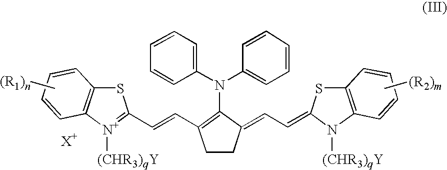

wherein R₁ and R₂ are the same and each is a halide, an ester of a carboxylic acid (—CO₂R), a carbamoyl group (—CONR), a sulfoxide, group, a sulfone group, a cyano group, a heterocyclic group or a nitro group and chosen such that the sum of the Hammett σₚₐᵣₐ and σₘₑₜₐ parameters of R₁ and R₂ is greater than 0.75; n and m each independently represents an integer 1 or 2; R₃ is hydrogen or an alkyl group; q is 3 or 4; Y is a sulfonic acid group; and X is a cation chosen so that the net charge of the entire molecule is zero. The most preferred infrared dyes are where n is 1 and R₁ and R₂ are each an ester of a carboxylic acid (—CO₂R where R is an alkyl or aryl group).

Specific examples of infrared dyes according to this invention include:

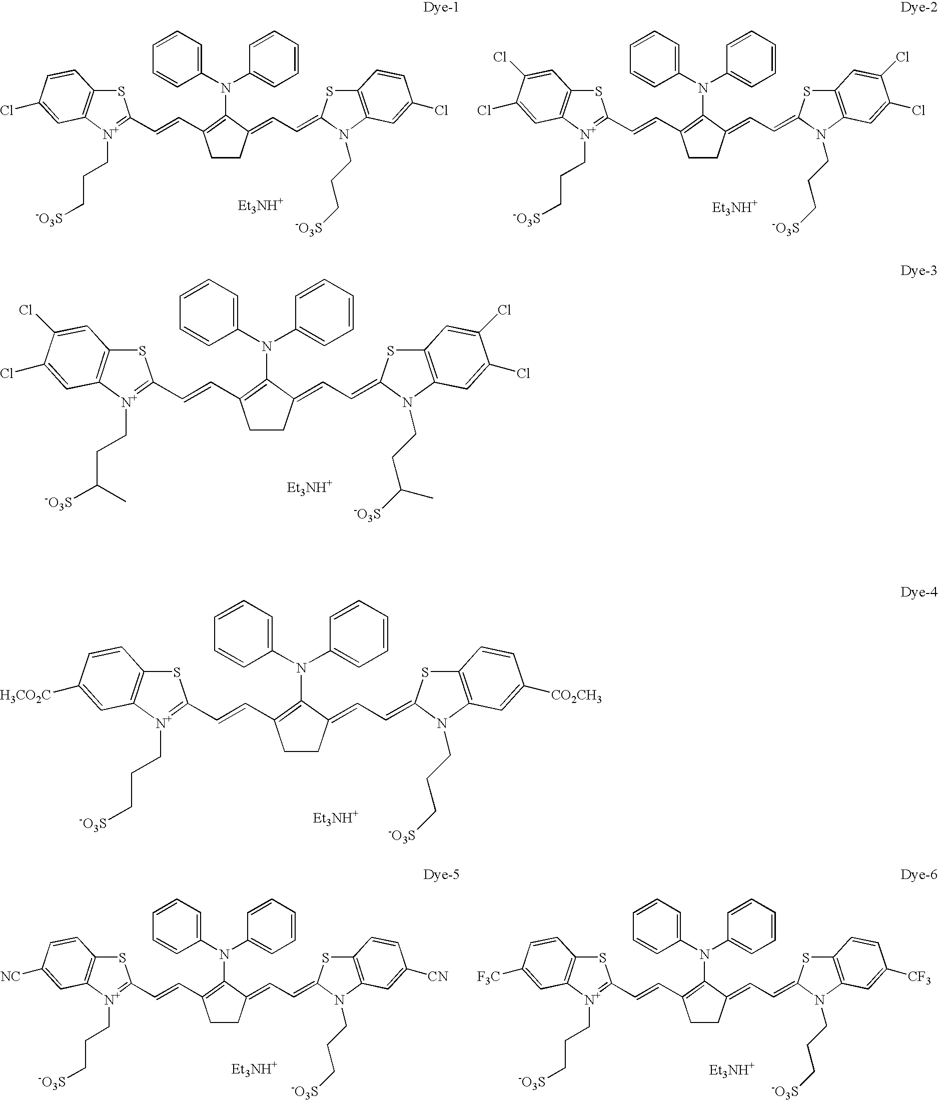

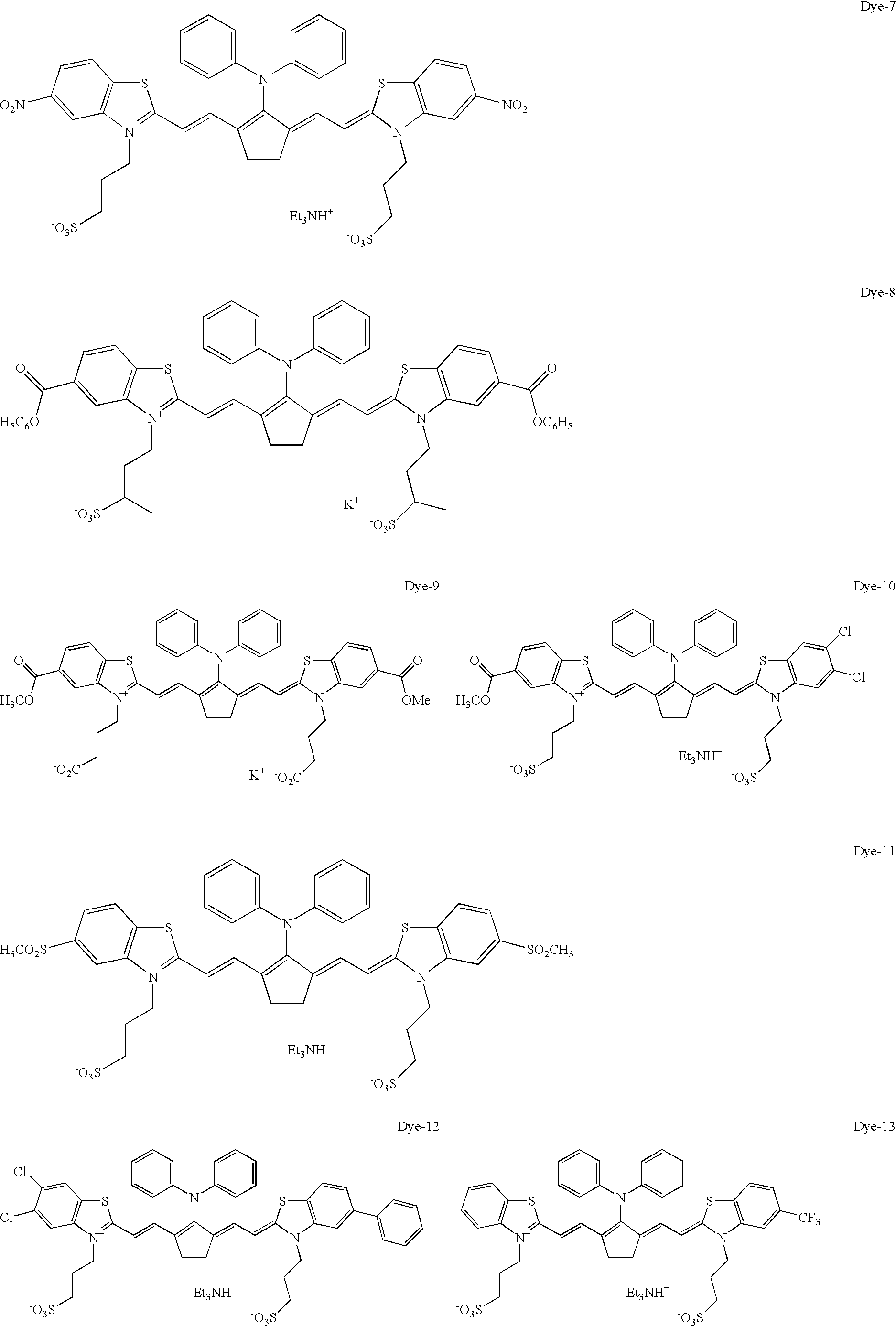

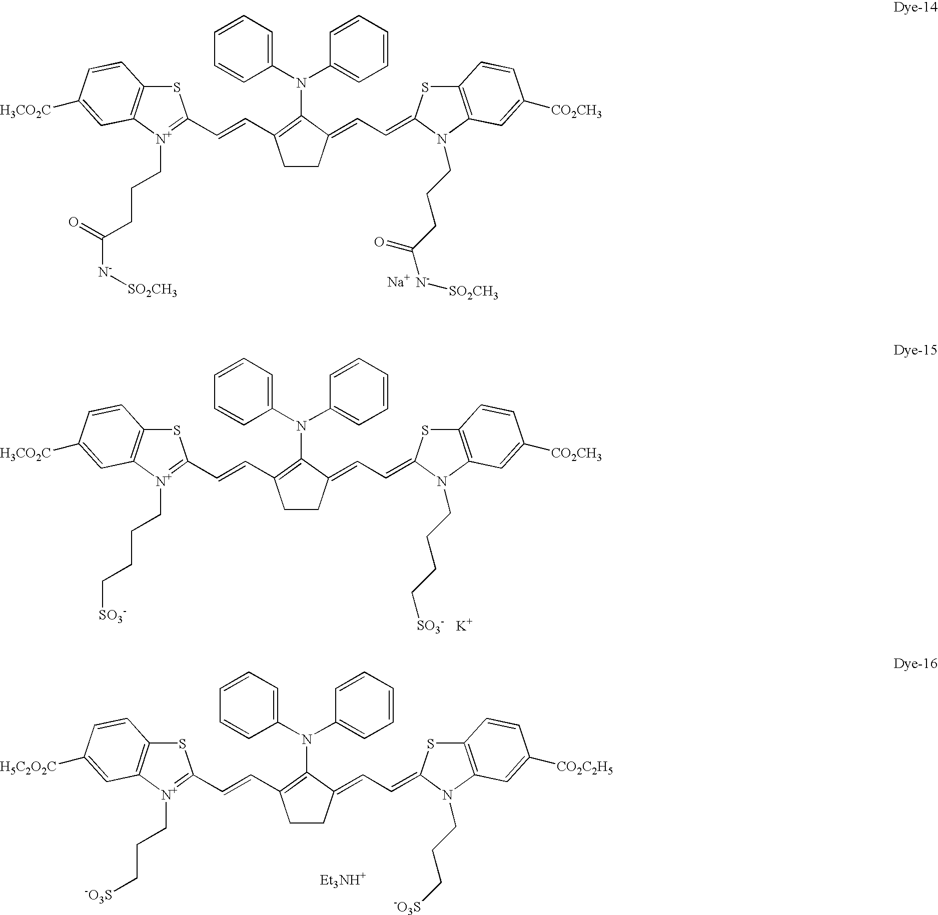

Unless otherwise specifically stated, use of the term “substituted” or “substituent” means any group or atom other than hydrogen. Additionally, when the term “group” is used, it means that when a substituent group contains a substitutable hydrogen, it is also intended to encompass not only the substituent's unsubstituted form, but also its form further substituted with any substituent group or groups as herein mentioned, so long as the substituent does not destroy properties necessary for photographic utility. Suitably, a substituent group may be halogen or may be bonded to the remainder of the molecule by an atom of carbon, silicon, oxygen, nitrogen, phosphorous, or sulfur. The substituent may be, for example, halogen, such as chlorine, bromine or fluorine; nitro; hydroxyl; cyano; carboxyl; or groups which may be further substituted, such as alkyl, including straight or branched chain or cyclic alkyl, such as methyl, trifluoromethyl, ethyl, t-butyl, 3-(2,4-di-t-pentylphenoxy)propyl, and tetradecyl; alkenyl, such as ethylene, 2-butene; alkoxy, such as methoxy, ethoxy, propoxy, butoxy, 2-methoxyethoxy, sec-butoxy, hexyloxy, 2-ethylhexyloxy, tetradecyloxy, 2-(2,4-di-t-pentylphenoxy)ethoxy, and 2-dodecyloxyethoxy; aryl such as phenyl, 4-t-butylphenyl, 2,4,6-trimethylphenyl, naphthyl; aryloxy, such as phenoxy, 2-methylphenoxy, alpha- or beta-naphthyloxy, and 4-tolyloxy; carbonamido, such as acetamido, benzamido, butyramido, tetradecanamido, alpha-(2,4-di-t-pentyl-phenoxy)acetamido, alpha-(2,4-di-t-pentylphenoxy)butyramido, alpha-(3-pentadecylphenoxy)-hexanamido, alpha-(4-hydroxy-3-t-butylphenoxy)-tetradecanamido, 2-oxo-pyrrolidin-1-yl, 2-oxo-5-tetradecylpyrrolin-1-yl, N-methyltetradecanamido, N-succinimido, N-phthalimido, 2,5-dioxo-1-oxazolidinyl, 3-dodecyl-2,5-dioxo-1-imidazolyl, and N-acetyl-N-dodecylamino, ethoxycarbonylamino, phenoxycarbonylamino, benzyloxycarbonylamino, hexadecyloxycarbonylamino, 2,4-di-t-butylphenoxycarbonylamino, phenylcarbonylamino, 2,5-(di-t-pentylphenyl)carbonylamino, p-dodecyl-phenylcarbonylamino, p-tolylcarbonylamino, N-methylureido, N,N-dimethylureido, N-methyl-N-dodecylureido, N-hexadecylureido, N,N-dioctadecylureido, N,N-dioctyl-N′-ethylureido, N-phenylureido, N,N-diphenylureido, N-phenyl-N-p-tolylureido, N-(m-hexadecylphenyl)ureido, N,N-(2,5-di-t-pentylphenyl)-N′-ethylureido, and t-butylcarbonamido; sulfonamido, such as methylsulfonamido, benzenesulfonamido, p-tolylsulfonamido, p-dodecylbenzenesulfonamido, N-methyltetradecylsulfonamido, N,N-dipropyl-sulfamoylamino, and hexadecylsulfonamido; sulfamoyl, such as N-methylsulfamoyl, N-ethylsulfamoyl, N,N-dipropylsulfamoyl, N-hexadecylsulfamoyl, N,N-dimethylsulfamoyl; N-[3-(dodecyloxy)propyl]sulfamoyl, N-[4-(2,4-di-t-pentylphenoxy)butyl]sulfamoyl, N-methyl-N-tetradecylsulfamoyl, and N-dodecylsulfamoyl; carbamoyl, such as N-methylcarbamoyl, N,N-dibutylcarbamoyl, N-octadecylcarbamoyl, N-[4-(2,4-di-t-pentylphenoxy)butyl]carbamoyl, N-methyl-N-tetradecylcarbamoyl, and N,N-dioctylcarbamoyl; acyl, such as acetyl, (2,4-di-t-amylphenoxy)acetyl, phenoxycarbonyl, p-dodecyloxyphenoxycarbonyl methoxycarbonyl, butoxycarbonyl, tetradecyloxycarbonyl, ethoxycarbonyl, benzyloxycarbonyl, 3-pentadecyloxycarbonyl, and dodecyloxycarbonyl; sulfonyl, such as methoxysulfonyl, octyloxysulfonyl, tetradecyloxysulfonyl, 2-ethylhexyloxysulfonyl, phenoxysulfonyl, 2,4-di-t-pentylphenoxysulfonyl, methylsulfonyl, octylsulfonyl, 2-ethylhexylsulfonyl, dodecylsulfonyl, hexadecylsulfonyl, phenylsulfonyl, 4-nonylphenylsulfonyl, and p-tolylsulfonyl; sulfonyloxy, such as dodecylsulfonyloxy, and hexadecylsulfonyloxy; sulfinyl, such as methylsulfinyl, octylsulfinyl, 2-ethylhexylsulfinyl, dodecylsulfinyl, hexadecylsulfinyl, phenylsulfinyl, 4-nonylphenylsulfinyl, and p-tolylsulfinyl; thio, such as ethylthio, octylthio, benzylthio, tetradecylthio, 2-(2,4-di-t-pentylphenoxy)ethylthio, phenylthio, 2-butoxy-5-t-octylphenylthio, and p-tolylthio; acyloxy, such as acetyloxy, benzoyloxy, octadecanoyloxy, p-dodecylamidobenzoyloxy, N-phenylcarbamoyloxy, N-ethylcarbamoyloxy, and cyclohexylcarbonyloxy; amine, such as phenylanilino, 2-chloroanilino, diethylamine, dodecylamine; imino, such as 1 (N-phenylimido)ethyl, N-succinimido or 3-benzylhydantoinyl; phosphate, such as dimethylphosphate and ethylbutylphosphate; phosphite, such as diethyl and dihexylphosphite; a heterocyclic group, a heterocyclic oxy group or a heterocyclic thio group, each of which may be substituted and which contain a 3- to 7-membered heterocyclic ring composed of carbon atoms and at least one hetero atom selected from the group consisting of oxygen, nitrogen and sulfur, such as 2-furyl, 2-thienyl, 2-benzimidazolyloxy or 2-benzothiazolyl; quaternary ammonium, such as triethylammonium; and silyloxy, such as trimethylsilyloxy.

If desired, the substituents may themselves be further substituted one or more times with the described substituent groups. The particular substituents used may be selected by those skilled in the art to attain the desired photographic properties for a specific application and can include, for example, hydrophobic groups, solubilizing groups, blocking groups, releasing or releasable groups, etc. When a molecule may have two or more substituents, the substituents may be joined together to form a ring such as a fused ring unless otherwise provided. Generally, the above groups and substituents thereof may include those having up to 48 carbon atoms, typically 1 to 36 carbon atoms and usually less than 24 carbon atoms, but greater numbers are possible depending on the particular substituents selected.

When the term “associated” is employed, it signifies that a reactive compound is in or adjacent to a specified layer where, during processing, it is capable of reacting with other components.

To control the migration of various components, it may be desirable to include a high molecular weight hydrophobe or “ballast” group in coupler molecules. Representative ballast groups include substituted or unsubstituted alkyl or aryl groups containing 8 to 42 carbon atoms. Representative substituents on such groups include alkyl, aryl, alkoxy, aryloxy, alkylthio, hydroxy, halogen, alkoxycarbonyl, aryloxycarbonyl, carboxy, acyl, acyloxy, amino, anilino, carbonamido, carbamoyl, alkylsulfonyl, arylsulfonyl, sulfonamido, and sulfamoyl groups wherein the substituents typically contain 1 to 42 carbon atoms. Such substituents can also be further substituted.

The photographic elements can be single color elements or multicolor elements. Multicolor elements contain image dye-forming units sensitive to each of the three primary regions of the spectrum. Each unit can comprise a single emulsion layer or multiple emulsion layers sensitive to a given region of the spectrum. The layers of the element, including the layers of the image-forming units, can be arranged in various orders as known in the art. In an alternative format, the emulsions sensitive to each of the three primary regions of the spectrum can be disposed as a single segmented layer.

A typical multicolor photographic element comprises a support bearing a cyan dye image-forming unit comprised of at least one red-sensitive silver halide emulsion layer having associated therewith at least one cyan dye-forming coupler, a magenta dye image-forming unit comprising at least one green-sensitive silver halide emulsion layer having associated therewith at least one magenta dye-forming coupler, and a yellow dye image-forming unit comprising at least one blue-sensitive silver halide emulsion layer having associated therewith at least one yellow dye-forming coupler. The element can contain additional layers, such as filter layers, interlayers, overcoat layers, subbing layers, and the like.

If desired, the photographic element can be used in conjunction with an applied magnetic layer as described in Research Disclosure, November 1992, Item 34390 published by Kenneth Mason Publications, Ltd., Dudley Annex, 12a North Street, Emsworth, Hampshire PO10 7DQ, ENGLAND, and as described in Hatsumi Kyoukai Koukai Gihou No. 94-6023, published Mar. 15, 1994, available from the Japanese Patent Office, the contents of which are incorporated herein by reference. When it is desired to employ the inventive materials in a small format film, Research Disclosure, June 1994, Item 36230, provides suitable embodiments. A particularly useful support for small format film is annealed polyethylenenaphthlate.

In the following discussion of suitable materials for use in the emulsions and elements of this invention, reference will be made to Research Disclosure, September 1996, Item 38957, available as described above, which will be identified hereafter by the term “Research Disclosure”. The contents of the Research Disclosure, including the patents and publications referenced therein, are incorporated herein by reference, and the Sections hereafter referred to are Sections of the Research Disclosure.

Except as provided, the silver halide emulsion containing elements employed in this invention can be either negative-working or positive-working as indicated by the type of processing instructions (i.e. color negative, reversal, or direct positive processing) provided with the element. More preferably the elements are negative working. Suitable emulsions and their preparation as well as methods of chemical and spectral sensitization are described in Sections I through V. Various additives such as UV dyes, brighteners, antifoggants, stabilizers, light absorbing and scattering materials, and physical property modifying addenda such as hardeners, coating aids, plasticizers, lubricants and matting agents are described, for example, in Sections II and VI through VIII. Color materials are described in Sections X through XIII. Suitable methods for incorporating couplers and dyes, including dispersions in organic solvents, are described in Section X(E). Scan facilitating is described in Section XIV. Supports, exposure, development systems, and processing methods and agents are described in Sections XV to XX. Certain desirable photographic elements and processing steps are described in Research Disclosure, Item 37038, February 1995.

The following discussion relates to any additional coupling species present in the film element in conjunction with the couplers of the invention.

Coupling-off groups are well known in the art. Such groups can determine the chemical equivalency of a coupler, i.e., whether it is a 2-equivalent or a 4-equivalent coupler, or modify the reactivity of the coupler. Such groups can advantageously affect the layer in which the coupler is coated, or other layers in the photographic recording material, by performing, after release from the coupler, functions such as dye formation, dye hue adjustment, development acceleration or inhibition, bleach acceleration or inhibition, electron transfer facilitation, color correction and the like.

The presence of hydrogen at the coupling site provides a 4-equivalent coupler, and the presence of another coupling-off group usually provides a 2-equivalent coupler. Representative classes of such coupling-off groups include, for example, chloro, alkoxy, aryloxy, hetero-oxy, sulfonyloxy, acyloxy, acyl, heterocyclyl such as oxazolidinyl or hydantoinyl, sulfonamido, mercaptotetrazole, benzothiazole, mercaptopropionic acid, phosphonyloxy, arylthio, and arylazo. These coupling-off groups are described in the art, for example, in U.S. Pat. Nos. 2,455,169, 3,227,551, 3,432,521, 3,476,563, 3,617,291, 3,880,661, 4,052,212 and 4,134,766; and in U.K. Patents and published application Nos. 1,466,728, 1,531,927, 1,533,039, 2,006,755A and 2,017,704A, the disclosures of which are incorporated herein by reference.

Image dye-forming couplers may be included in the element such as couplers that form cyan dyes upon reaction with oxidized color developing agents which are described in such representative patents and publications as: U.S. Pat. Nos. 2,367,531, 2,423,730, 2,474,293, 2,772,162, 2,895,826, 3,002,836, 3,034,892, 3,041,236, 4,333,999, 4,883,746 and “Farbkuppler-eine LiteratureUbersicht,” published in Agfa Mitteilungen, Band III, pp. 156-175 (1961). Preferably such couplers are phenols and naphthols that form cyan dyes on reaction with oxidized color developing agent.

Couplers that form magenta dyes upon reaction with oxidized color developing agent are described in such representative patents and publications as: U.S. Pat. Nos. 2,311,082, 2,343,703, 2,369,489, 2,600,788, 2,908,573, 3,062,653, 3,152,896, 3,519,429, 3,758,309, 4,540,654, and “Farbkuppler-eine LiteratureUbersicht,” published in Agfa Mitteilungen, Band III, pp. 126-156 (1961). Preferably such couplers are pyrazolones, pyrazolotriazoles, or pyrazolobenzimidazoles that form magenta dyes upon reaction with oxidized color developing agents.

Couplers that form yellow dyes upon reaction with oxidized and color developing agent are described in such representative patents and publications as: U.S. Pat. Nos. 2,298,443, 2,407,210, 2,875,057, 3,048,194, 3,265,506, 3,447,928, 4,022,620, 4,443,536, and “Farbkuppler-eine LiteratureUbersicht,” published in Agfa Mitteilungen, Band III, pp. 112-126 (1961). Such couplers are typically open chain ketomethylene compounds.

Couplers that form colorless products upon reaction with oxidized color developing agent are described in such representative patents as: U.K. Patent No. 861,138; U.S. Pat. Nos. 3,632,345, 3,928,041, 3,958,993 and 3,961,959. Typically such couplers are cyclic carbonyl containing compounds that form colorless products on reaction with an oxidized color developing agent.

Couplers that form black dyes upon reaction with oxidized color developing agent are described in such representative patents as U.S. Pat. Nos. 1,939,231; 2,181,944; 2,333,106; and 4,126,461; German OLS No. 2,644,194 and German OLS No. 2,650,764. Typically, such couplers are resorcinols or m-aminophenols that form black or neutral products on reaction with oxidized color developing agent.

In addition to the foregoing, so-called “universal” or “washout” couplers may be employed. These couplers do not contribute to image dye-formation. Thus, for example, a naphthol having an unsubstituted carbamoyl or one substituted with a low molecular weight substituent at the 2- or 3-position may be employed. Couplers of this type are described, for example, in U.S. Pat. Nos. 5,026,628, 5,151,343, and 5,234,800.

It may be useful to use a combination of couplers any of which may contain known ballasts or coupling-off groups such as those described in U.S. Pat. No. 4,301,235; U.S. Pat. No. 4,853,319 and U.S. Pat. No. 4,351,897. The coupler may contain solubilizing groups such as described in U.S. Pat. No. 4,482,629. The coupler may also be used in association with “wrong” colored couplers (e.g. to adjust levels of interlayer correction) and, in color negative applications, with masking couplers such as those described in EP 213,490; Japanese Published Application 58-172,647; U.S. Pat. Nos. 2,983,608; 4,070,191; and 4,273,861; German Applications DE 2,706,117 and DE 2,643,965; U.K. Patent 1,530,272; and Japanese Application 58-113935. The masking couplers maybe shifted or blocked, if desired.

Typically, couplers are incorporated in a silver halide emulsion layer in a mole ratio to silver of 0.05 to 1.0 and generally 0.1 to 0.5. Usually the couplers are dispersed in a high-boiling organic solvent in a weight ratio of solvent to coupler of 0.1 to 10.0 and typically 0.1 to 2.0 although dispersions using no permanent coupler solvent are sometimes employed.

The invention materials may be used in association with materials that accelerate or otherwise modify the processing steps e.g. of bleaching or fixing to improve the quality of the image. Bleach accelerator releasing couplers such as those described in EP 193,389; EP 301,477; U.S. Pat. No. 4,163,669; U.S. Pat. No. 4,865,956; and U.S. Pat. No. 4,923,784, may be useful. Also contemplated is use of the compositions in association with nucleating agents, development accelerators or their precursors (UK Patent 2,097,140; U.K. Patent 2,131,188); electron transfer agents (U.S. Pat. No. 4,859,578; U.S. Pat. No. 4,912,025); antifogging and anti color-mixing agents such as derivatives of hydroquinones, aminophenols, amines, gallic acid; catechol; ascorbic acid; hydrazides; sulfonamidophenols; and non color-forming couplers.

The invention materials may also be used in combination with filter dye layers comprising colloidal silver sol or yellow, cyan, and/or magenta filter dyes, either as oil-in-water dispersions, latex dispersions or as solid particle dispersions. Additionally, they may be used with “smearing” couplers (e.g., as described in U.S. Pat. No. 4,366,237; EP 96,570; U.S. Pat. No. 4,420,556; and U.S. Pat. No. 4,543,323.) Also, the compositions may be blocked or coated in protected form as described, for example, in Japanese Application 61/258,249 or U.S. Pat. No. 5,019,492.

The invention materials may further be used in combination with image-modifying compounds such as “Developer Inhibitor-Releasing” compounds (DIR's). DIR's useful in conjunction with the compositions of the invention are known in the art and examples are described in U.S. Pat. Nos. 3,137,578; 3,148,022; 3,148,062; 3,227,554; 3,384,657; 3,379,529; 3,615,506; 3,617,291; 3,620,746; 3,701,783; 3,733,201; 4,049,455; 4,095,984; 4,126,459; 4,149,886; 4,150,228; 4,211,562; 4,248,962; 4,259,437; 4,362,878; 4,409,323; 4,477,563; 4,782,012; 4,962,018; 4,500,634; 4,579,816; 4,607,004; 4,618,571; 4,678,739; 4,746,600; 4,746,601; 4,791,049; 4,857,447; 4,865,959; 4,880,342; 4,886,736; 4,937,179; 4,946,767; 4,948,716; 4,952,485; 4,956,269; 4,959,299; 4,966,835; 4,985,336 as well as in patent publications GB 1,560,240; GB 2,007,662; GB 2,032,914; GB 2,099,167; DE 2,842,063, DE 2,937,127; DE 3,636,824; DE 3,644,416 as well as the following European Patent Publications: 272,573; 335,319; 336,411; 346, 899; 362, 870; 365,252; 365,346; 373,382; 376,212; 377,463; 378,236; 384,670; 396,486; 401,612; 401,613.

Such compounds are also disclosed in “Developer-Inhibitor-Releasing (DIR) Couplers for Color Photography,” C. R. Barr, J. R. Thirtle and P. W. Vittum in Photographic Science and Engineering, Vol. 13, p. 174 (1969), incorporated herein by reference. Generally, the developer inhibitor-releasing (DIR) couplers include a coupler moiety and an inhibitor coupling-off moiety (IN). The inhibitor-releasing couplers may be of the time-delayed type (DIAR couplers) which also include a timing moiety or chemical switch which produces a delayed release of inhibitor. Examples of typical inhibitor moieties are: oxazoles, thiazoles, diazoles, triazoles, oxadiazoles, thiadiazoles, oxathiazoles, thiatriazoles, benzotriazoles, tetrazoles, benzimidazoles, indazoles, isoindazoles, mercaptotetrazoles, selenotetrazoles, mercaptobenzothiazoles, selenobenzothiazoles, mercaptobenzoxazoles, selenobenzoxazoles, mercaptobenzimidazoles, selenobenzimidazoles, benzodiazoles, mercaptooxazoles, mercaptothiadiazoles, mercaptothiazoles, mercaptotriazoles, mercaptooxadiazoles, mercaptodiazoles, mercaptooxathiazoles, telleurotetrazoles or benzisodiazoles. In a preferred embodiment, the inhibitor moiety or group is selected from the following formulas:

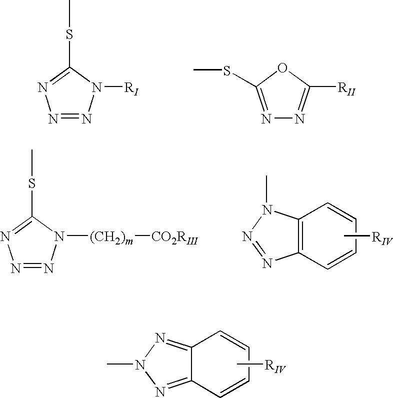

wherein R_{I} is selected from the group consisting of straight and branched alkyls of from 1 to about 8 carbon atoms, benzyl, phenyl, and alkoxy groups and such groups containing none, one or more than one such substituent; R_{II} is selected from R_{I} and —SR_{I}; R_{III} is a straight or branched alkyl group of from 1 to about 5 carbon atoms and m is from 1 to 3; and R_{IV} is selected from the group consisting of hydrogen, halogens and alkoxy, phenyl and carbonamido groups, —COOR_{V} and —NHCOOR_{V} wherein R_{V} is selected from substituted and unsubstituted alkyl and aryl groups.

Although it is typical that the coupler moiety included in the developer inhibitor-releasing coupler forms an image dye corresponding to the layer in which it is located, it may also form a different color as one associated with a different film layer. It may also be useful that the coupler moiety included in the developer inhibitor-releasing coupler forms colorless products and/or products that wash out of the photographic material during processing (so-called “universal” couplers).

A compound such as a coupler may release a PUG directly upon reaction of the compound during processing, or indirectly through a timing or linking group. A timing group produces the time-delayed release of the PUG such groups using an intramolecular nucleophilic substitution reaction (U.S. Pat. No. 4,248,962); groups utilizing an electron transfer reaction along a conjugated system (U.S. Pat. Nos. 4,409,323; 4,421,845; 4,861,701, Japanese Applications 57-188035; 58-98728; 58-209736; 58-209738); groups that function as a coupler or reducing agent after the coupler reaction (U.S. Pat. No. 4,438,193; U.S. Pat. No. 4,618,571) and groups that combine the features describe above. It is typical that the timing group is of one of the formulas:

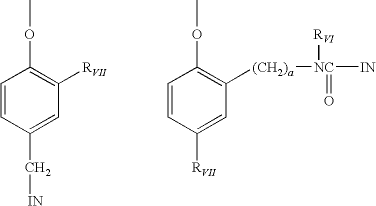

wherein IN is the inhibitor moiety, R_{VII} is selected from the group consisting of nitro, cyano, alkylsulfonyl; sulfamoyl; and sulfonamido groups; a is 0 or 1; and R_{VI} is selected from the group consisting of substituted and unsubstituted alkyl and phenyl groups. The oxygen atom of each timing group is bonded to the coupling-off position of the respective coupler moiety of the DIAR.

The timing or linking groups may also function by electron transfer down an unconjugated chain. Linking groups are known in the art under various names. Often they have been referred to as groups capable of utilizing a hemiacetal or iminoketal cleavage reaction or as groups capable of utilizing a cleavage reaction due to ester hydrolysis such as U.S. Pat. No. 4,546,073. This electron transfer down an unconjugated chain typically results in a relatively fast decomposition and the production of carbon dioxide, formaldehyde, or other low molecular weight by-products. The groups are exemplified in EP 464,612, EP 523,451, U.S. Pat. No. 4,146,396, Japanese Kokai 60-249148 and 60-249149.

Suitable developer inhibitor-releasing couplers for use in the present invention include, but are not limited to, the following:

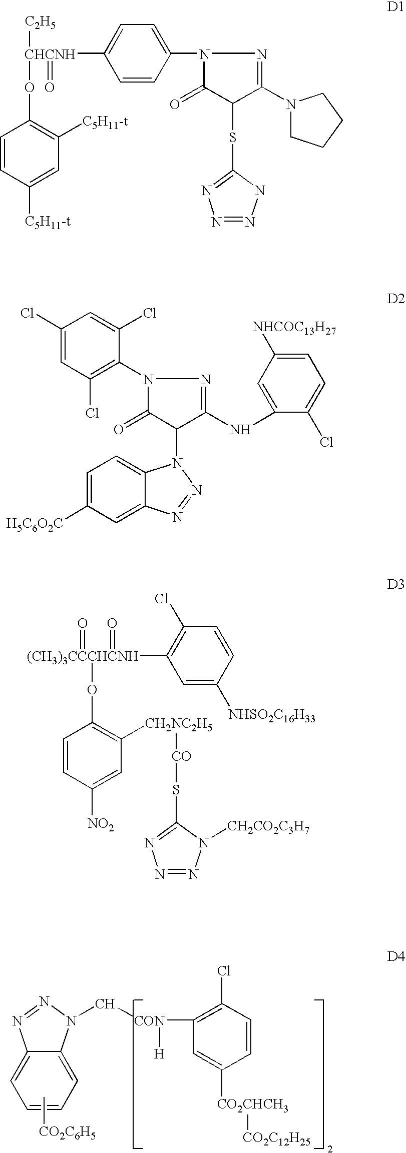

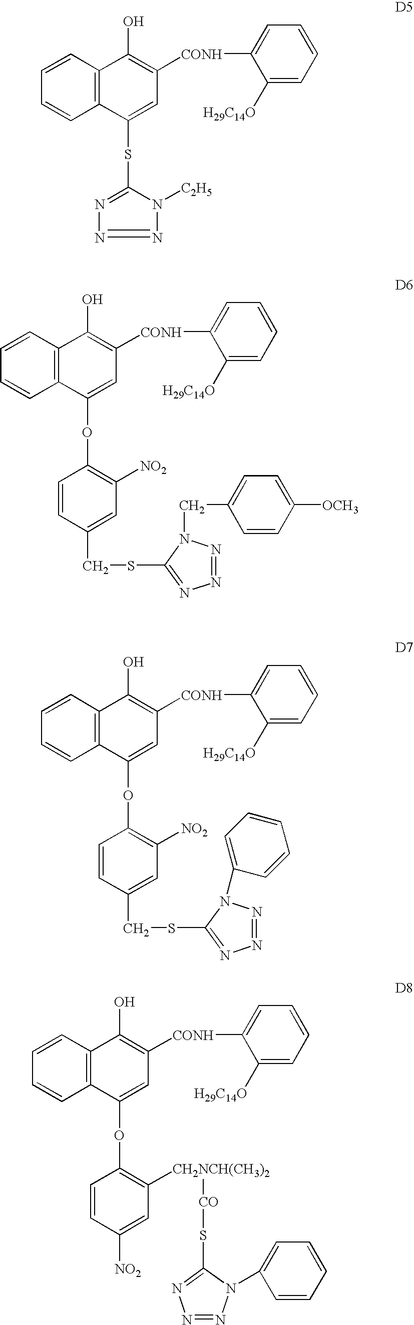

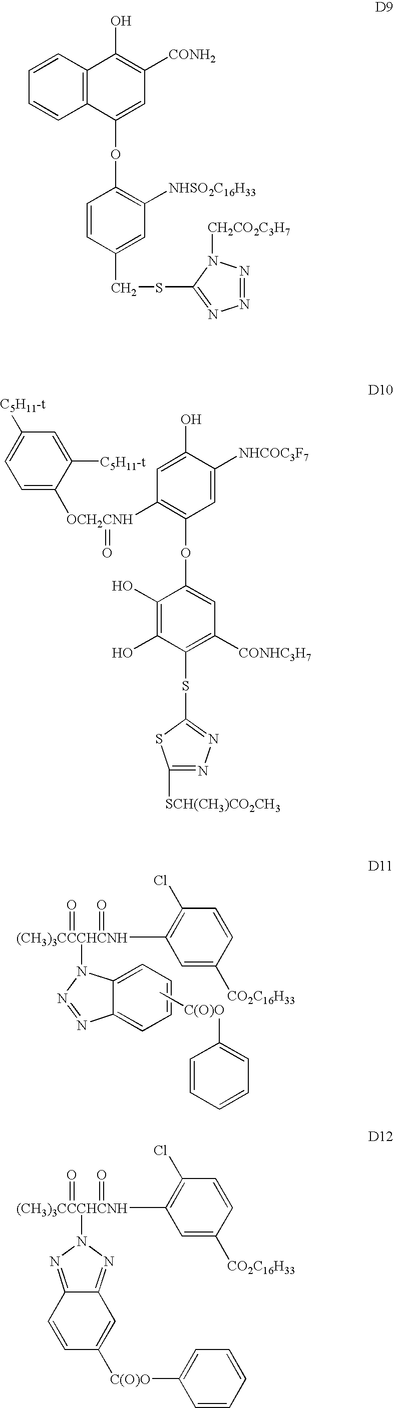

Moreover, speed enhancing materials such as those described in U.S. Pat. No. 6,455,242; U.S. Pat. No. 6,426,180; U.S. Pat. No. 6,350,564 and U.S. Pat. No. 6,319,660 maybe used.

Unless indicated otherwise, compounds used directly in a photographic element can be added to a mixture containing silver halide before coating or, more suitably, be mixed with the silver halide just prior to or during coating. In either case, additional components like couplers, doctors, surfactants, hardeners and other materials that are typically present in such solutions may also be present at the same time. Coupling materials are generally not water-soluble and cannot be added directly to the solution. They may be added directly if dissolved in an organic water miscible solution such as methanol, acetone or the like or more preferably as a dispersion. A dispersion incorporates the material in a stable, finely divided state in a hydrophobic organic solvent (often referred to as a coupler solvent or permanent solvent) that is stabilized by suitable surfactants and surface active agents usually in combination with a binder or matrix such as gelatin. The dispersion may contain one or more permanent solvents that dissolve the material and maintain it in a liquid state. Some examples of suitable permanent solvents are tricresylphosphate, N,N-diethyllauramide, N,N-dibutyllauramide, p-dodecylphenol, dibutylphthalate, di-n-butyl sebacate, N-n-butylacetanilide, 9-octadecen-1-ol, ortho-methylphenyl benzoate, trioctylamine and 2-ethylhexylphosphate. Preferred classes of solvents are carbonamides, phosphates, alcohols and esters. When a solvent is present, it is preferred that the weight ratio of compound to solvent be at least 1 to 0.5, or most preferably, at least 1 to 1. The dispersion may require an auxiliary coupler solvent initially to dissolve the component but this is removed afterwards, usually either by evaporation or by washing with additional water. Some examples of suitable auxiliary coupler solvents are ethyl acetate, cyclohexanone and 2-(2-butoxyethoxy)ethyl acetate. The dispersion may also be stabilized by addition of polymeric materials to form stable latexes. Examples of suitable polymers for this use generally contain water-solubilizing groups or have regions of high hydrophilicity. Some examples of suitable dispersing agents or surfactants are Alkanol XC or saponin. The materials used in the invention may also be dispersed as an admixture with another component of the system such as a coupler or an oxidized developer scavenger so that both are present in the same oil droplet. It is also possible to incorporate the materials of the invention as a solid particle dispersion; that is, a slurry or suspension of finely ground (through mechanical means) compound. These solid particle dispersions may be additionally stabilized with surfactants and/or polymeric materials as known in the art. Also, additional permanent solvent may be added to the solid particle dispersion to help increase activity.

The silver halide used in the photographic elements may be silver iodobromide, silver bromide, silver chloride, silver chlorobromide, silver chloroiodobromide, and the like. The grain size of the silver halide may have any distribution known to be useful in photographic compositions, and may be either polydispersed or monodispersed.

The silver halide grains to be used in the invention may be prepared according to methods known in the art, such as those described in Research Disclosure I and The Theory of the Photogaphic Process, 4^{th} edition, T. H. James, editor, Macmillan Publishing Co., New York, 1977. These include methods such as ammoniacal emulsion making, neutral or acidic emulsion making, and others known in the art. These methods generally involve mixing a water soluble silver salt with a water soluble halide salt in the presence of a protective colloid, and controlling the temperature, pAg, pH values, etc., at suitable values during formation of the silver halide by precipitation.

Especially useful in this invention are radiation-sensitive tabular grain silver halide emulsions. Tabular grains are silver halide grains having parallel major faces and an aspect ratio of at least 2, where aspect ratio is the ratio of grain equivalent circular diameter (ECD) divided by grain thickness (t). The equivalent circular diameter of a grain is the diameter of a circle having an average equal to the projected area of the grain. A tabular grain emulsion is one in which tabular grains account for greater than 50 percent of total grain projected area. In preferred tabular grain emulsions tabular grains account for at least 70 percent of total grain projected area and optimally at least 90 percent of total grain projected area. It is possible to prepare tabular grain emulsions in which substantially all (>97%) of the grain projected area is accounted for by tabular grains. The non-tabular grains in a tabular grain emulsion can take any convenient conventional form. When coprecipitated with the tabular grains, the non-tabular grains typically exhibit a silver halide composition as the tabular grains.

The tabular grain emulsions can be either high bromide or high chloride emulsions. High bromide emulsions are those in which silver bromide accounts for greater than 50 mole percent of total halide, based on silver. High chloride emulsions are those in which silver chloride accounts for greater than 50 mole percent of total halide, based on silver. Silver bromide and silver chloride both form a face centered cubic crystal lattice structure. This silver halide crystal lattice structure can accommodate all proportions of bromide and chloride ranging from silver bromide with no chloride present to silver chloride with no bromide present. Thus, silver bromide, silver chloride, silver bromochloride and silver chlorobromide tabular grain emulsions are all specifically contemplated. In naming grains and emulsions containing two or more halides, the halides are named in order of ascending concentrations. Usually high chloride and high bromide grains that contain bromide or chloride, respectively, contain the lower level halide in a more or less uniform distribution. However, non-uniform distributions of chloride and bromide are known, as illustrated by Maskasky U.S. Pat. Nos. 5,508,160 and 5,512,427 and Delton U.S. Pat. Nos. 5,372,927 and 5,460,934, the disclosures of which are here incorporated by reference.

It is recognized that the tabular grains can accommodate iodide up to its solubility limit in the face centered cubic crystal lattice structure of the grains. The solubility limit of iodide in a silver bromide crystal lattice structure is approximately 40 mole percent, based on silver. The solubility limit of iodide in a silver chloride crystal lattice structure is approximately 11 mole percent, based on silver. The exact limits of iodide incorporation can be somewhat higher or lower, depending upon the specific technique employed for silver halide grain preparation. In practice, useful photographic performance advantages can be realized with iodide concentrations as low as 0.1 mole percent, based on silver. It is usually preferred to incorporate at least 0.5 (optimally at least 1.0) mole percent iodide, based on silver. Only low levels of iodide are required to realize significant emulsion speed increases. Higher levels of iodide are commonly incorporated to achieve other photographic effects, such as interimage effects. Overall iodide concentrations of up to 20 mole percent, based on silver, are well known, but it is generally preferred to limit iodide to 15 mole percent, more preferably 10 mole percent, or less, based on silver. Higher than needed iodide levels are generally avoided, since it is well recognized that iodide slows the rate of silver halide development.

Iodide can be uniformly or non-uniformly distributed within the tabular grains. Both uniform and non-uniform iodide concentrations are known to contribute to photographic speed. For maximum speed it is common practice to distribute iodide over a large portion of a tabular grain while increasing the local iodide concentration within a limited portion of the grain. It is also common practice to limit the concentration of iodide at the surface of the grains. Preferably the surface iodide concentration of the grains is less than 5 mole percent, based on silver. Surface iodide is the iodide that lies within 0.02 nm of the grain surface.

With iodide incorporation in the grains, the high chloride and high bromide tabular grain emulsions within the contemplated of the invention extend to silver iodobromide, silver iodochloride, silver iodochlorobromide and silver iodobromochloride tabular grain emulsions.

When tabular grain emulsions are spectrally sensitized, as herein contemplated, it is preferred to limit the average thickness of the tabular grains to less than 0.3 μm. Most preferably the average thickness of the tabular grains is less than 0.2 μm. In a specific preferred form the tabular grains are ultrathin—that is, their average thickness is less than 0.07 μm.

The useful average grain ECD of a tabular grain emulsion can range up to about 15 μm. Except for a very few high speed applications, the average grain ECD of a tabular grain emulsion is conventionally less than 10 μm, with the average grain ECD for most tabular grain emulsions being less than 5 μm.

The average aspect ratio of the tabular grain emulsions can vary widely, since it is quotient of ECD divided by grain thickness. Most tabular grain emulsions have average aspect ratios of greater than 5, with high (>8) average aspect ratio emulsions being generally preferred. Average aspect ratios ranging up to 50 are common, with average aspect ratios ranging up to 100 and even higher, being known.

The tabular grains can have parallel major faces that lie in either {100} or {111} crystal lattice planes. In other words, both {111} tabular grain emulsions and {100} tabular grain emulsions are within the specific contemplation of this invention. The {111} major faces of {111} tabular grains appear triangular or hexagonal in photomicrographs while the {100} major faces of {100} tabular grains appear square or rectangular.

High chloride {111} tabular grain emulsions are illustrated by Wey U.S. Pat. No. 4,399,215, Wey et al U.S. Pat. No. 4,414,306, Maskasky U.S. Pat. Nos. 4,400,463, 4,713,323, 5,061,617, 5,178,997, 5,183,732, 5,185,239, 5,399,478 and 5,411,852, Maskasky et al U.S. Pat. Nos. 5,176,992 and 5,178,998, Takada et al U.S. Pat. No. 4,783,398, Nishikawa et al U.S. Pat. No. 4,952,508, Ishiguro et al U.S. Pat. No. 4,983,508, Tufano et al U.S. Pat. No. 4,804,621, Maskasky and Chang U.S. Pat. No. 5,178,998, and Chang et al U.S. Pat. No. 5,252,452. Ultrathin high chloride {111} tabular grain emulsions are illustrated by Maskasky U.S. Pat. Nos. 5,271,858 and 5,389,509.

Since silver chloride grains are most stable in terms of crystal shape with {100} crystal faces, it is common practice to employ one or more grain growth modifiers during the formation of high chloride {111} tabular grain emulsions. Typically the grain growth modifier is displaced prior to or during subsequent spectral sensitization, as illustrated by Jones et al U.S. Pat. No. 5,176,991 and Maskasky U.S. Pat. Nos. 5,176,992, 5,221,602, 5,298,387 and 5,298,388, the disclosures of which are here incorporated by reference.

Preferred high chloride tabular grain emulsions are {100} tabular grain emulsions, as illustrated by the following patents, here incorporated by reference: Maskasky U.S. Pat. Nos. 5,264,337, 5,292,632, 5,275,930, 5,607,828 and 5,399,477, House et al U.S. Pat. No. 5,320,938, Brust et al U.S. Pat. No. 5,314,798, Szajewski et al U.S. Pat. No. 5,356,764, Chang et al U.S. Pat. Nos. 5,413,904, 5,663,041, and 5,744,297, Budz et al U.S. Pat. No. 5,451,490, Reed et al U.S. Pat. No. 5,695,922, Oyamada U.S. Pat. No. 5,593,821, Yamashita et al U.S. Pat. Nos. 5,641,620 and 5,652,088, Saitou et al U.S. Pat. No. 5,652,089, and Oyamada et al U.S. Pat. No. 5,665,530. Ultrathin high chloride {100} tabular grain emulsions can be prepared by nucleation in the presence of iodide, following the teaching of House et al and Chang et al, cited above. Since high chloride {100} tabular grains have {100} major faces and are, in most instances, entirely bounded by {100} grain faces, these grains exhibit a high degree of grain shape stability and do not require the presence of any grain growth modifier for the grains to remain in a tabular form following their precipitation.

In their most widely used form tabular grain emulsions are high bromide {111} tabular grain emulsions. Such emulsions are illustrated by Kofron et al U.S. Pat. No. 4,439,520, Wilgus et al U.S. Pat. No. 4,434,226, Solberg et al U.S. Pat. No. Patent 4,433,048, Maskasky U.S. Pat. Nos. 4,435,501, 4,463,087 4,173,320 and 5,411,851 5,418,125, 5,492,801, 5,604,085, 5,620,840, 5,693,459, 5,733,718, Daubendiek et al U.S. Pat. Nos. 4,414,310 and 4,914,014, Sowinski et al U.S. Pat. No. 4,656,122, Piggin et al U.S. Pat. Nos. 5,061,616 and 5,061,609, Tsaur et al U.S. Pat. Nos. 5,147,771, '772, '773, 5,171,659 and 5,252,453, Black et al U.S. Pat. Nos. 5,219,720 and 5,334,495, Delton U.S. Pat. Nos. 5,310,644, 5,372,927 and 5,460,934, Wen U.S. Pat. No. 5,470,698, Fenton et al U.S. Pat. No. 5,476,760, Eshelman et al U.S. Pat. Nos. 5,612,175, 5,612,176 and 5,614,359, and Irving et al U.S. Pat. Nos. 5,695,923, 5,728,515 and 5,667,954, Bell et al U.S. Pat. No. 5,132,203, Brust U.S. Pat. Nos. 5,248,587 and 5,763,151,. Chaffee et al U.S. Pat. No. 5,358,840, Deaton et al U.S. Pat. No. 5,726,007, King et al U.S. Pat. No. 5,518,872, Levy et al U.S. Pat. No. 5,612,177, Mignot et al U.S. Pat. No. 5,484,697, Olm et al U.S. Pat. No. 5,576,172, Reed et al U.S. Pat. Nos. 5,604,086 and 5,698,387.

Ultrathin high bromide {111} tabular grain emulsions are illustrated by Daubendiek et al U.S. Pat. Nos. 4,672,027, 4,693,964, 5,494,789, 5,503,971 and 5,576,168, Antoniades et al U.S. Pat. No. 5,250,403, Olm et al U.S. Pat. No. 5,503,970, Deaton et al U.S. Pat. No. 5,582,965, and Maskasky U.S. Pat. No. 5,667,955. High bromide {100} tabular grain emulsions are illustrated by Mignot U.S. Pat. Nos. 4,386,156 and 5,386,156.

High bromide {100} tabular grain emulsions are known, as illustrated by Mignot U.S. Pat. No. 4,386,156 and Gourlaouen et al U.S. Pat. No. 5,726,006.

In many of the patents listed above (starting with Kofron et al, Wilgus et al and Solberg et al, cited above) speed increases without accompanying increases in granularity are realized by the rapid (a.k.a. dump) addition of iodide for a portion of grain growth. Chang et al U.S. Pat. No. 5,314,793 correlates rapid iodide addition with crystal lattice disruptions observable by stimulated X-ray emission profiles.

Localized peripheral incorporations of higher iodide concentrations can also be created by halide conversion. By controlling the conditions of halide conversion by iodide, differences in peripheral iodide concentrations at the grain corners and elsewhere along the edges can be realized. For example, Fenton et al U.S. Pat. No. 5,476,76 discloses lower iodide concentrations at the corners of the tabular grains than elsewhere along their edges. Jagannathan et al U.S. Pat. Nos. 5,723,278 and 5,736,312 disclose halide conversion by iodide in the corner regions of tabular grains.

Crystal lattice dislocations, although seldom specifically discussed, are a common occurrence in tabular grains. For example, examinations of the earliest reported high aspect ratio tabular grain emulsions (e.g., those of Kofron et al, Wilgus et al and Solberg et al, cited above) reveal high levels of crystal lattice dislocations. Black et al U.S. Pat. No. 5,709,988 correlates the presence of peripheral crystal lattice dislocations in tabular grains with improved speed-granularity relationships. Ikeda et al U.S. Pat. No. 4,806,461 advocates employing tabular grain emulsions in which at least 50 percent of the tabular grains contain 10 or more dislocations. For improving speed-granularity characteristics, it is preferred that at least 70 percent and optimally at least 90 percent of the tabular grains contain 10 or more peripheral crystal lattice dislocations.

The silver halide emulsion may comprise tabular silver halide grains having surface chemical sensitization sites including at least one silver salt forming epitaxial junction with the tabular grains and being restricted to those portions of the tabular grains located nearest peripheral edges.

The silver halide tabular grains of the photographic material may be prepared with a maximum surface iodide concentration along the edges and a lower surface iodide concentration within the corners than elsewhere along the edges.

In the course of grain precipitation one or more dopants (grain occlusions other than silver and halide) can be introduced to modify grain properties. For example, any of the various conventional dopants disclosed in Research Disclosure, Item 38957, Section I. Emulsion grains and their preparation, sub-section G. Grain modifying conditions and adjustments, paragraphs (3), (4) and (5), can be present in the emulsions of the invention. Especially useful dopants are disclosed by Marchetti et al., U.S. Pat. No. 4,937,180, and Johnson et al., U.S. Pat. No. 5,164,292. In addition it is specifically contemplated to dope the grains with transition metal hexacoordination complexes containing one or more organic ligands, as taught by Olm et al U.S. Pat. No. 5,360,712, the disclosure of which is here incorporated by reference.

It is specifically contemplated to incorporate in the face centered cubic crystal lattice of the grains a dopant capable of increasing imaging speed by forming a shallow electron trap (hereinafter also referred to as a SET) as discussed in Research Disclosure Item 36736 published November 1994, here incorporated by reference.

SET dopants are known to be effective to reduce reciprocity failure. In particular the use of Ir⁺³ or Ir⁺⁴ hexacoordination complexes as SET dopants is advantageous.

Iridium dopants that are ineffective to provide shallow electron traps (non-SET dopants) can also be incorporated into the grains of the silver halide grain emulsions to reduce reciprocity failure.

The contrast of the photographic element can be further increased by doping the grains with a hexacoordination complex containing a nitrosyl or thionitrosyl ligand (NZ dopants) as disclosed in McDugle et al U.S. Pat. No. 4,933,272, the disclosure of which is here incorporated by reference.

The emulsions can be surface-sensitive emulsions, i.e., emulsions that form latent images primarily on the surfaces of the silver halide grains, or the emulsions can form internal latent images predominantly in the interior of the silver halide grains. The emulsions can be negative-working emulsions, such as surface-sensitive emulsions or unfogged internal latent image-forming emulsions, or direct-positive emulsions of the unfogged, internal latent image-forming type, which are positive-working when development is conducted with uniform light exposure or in the presence of a nucleating agent. Tabular grain emulsions of the latter type are illustrated by Evans et al. U.S. Pat. No. 4,504,570.

Photographic elements can be exposed to actinic radiation, typically in the visible region of the spectrum, to form a latent image and can then be processed to form a visible dye image. Processing to form a visible dye image includes the step of contacting the element with a color developing agent to reduce developable silver halide and oxidize the color developing agent. Oxidized color developing agent in turn reacts with the coupler to yield a dye.

With negative-working silver halide, the processing step described above provides a negative image. One type of such element, referred to as a color negative film, is designed for image capture. Preferably the materials of the invention are color negative films. Speed (the sensitivity of the element to low light conditions) is usually critical to obtaining sufficient image in such elements. Such elements are typically silver bromoiodide emulsions coated on a transparent support and are sold packaged with instructions to process in known color negative processes such as the Kodak C-41 process as described in The British Journal of Photography Annual of 1988, pages 191-198. If a color negative film element is to be subsequently employed to generate a viewable projection print as for a motion picture, a process such as the Kodak ECN-2 process described in the H-24 Manual available from Eastman Kodak Co. may be employed to provide the color negative image on a transparent support. Color negative development times are typically 3′ 15″ or less and desirably 90 or even 60 seconds or less.

The photographic element of the invention can be incorporated into exposure structures intended for repeated use or exposure structures intended for limited use, variously referred to by names such as “one time use camera”, “single use cameras”, “lens with film”, or “photosensitive material package units”.

Another type of color negative element is a color print. Such an element is designed to receive an image optically printed from an image capture color negative element. A color print element may be provided on a reflective support for reflective viewing (e.g., a snapshot) or on a transparent support for projection viewing as in a motion picture. Elements destined for color reflection prints are provided on a reflective support, typically paper, employ silver chloride emulsions, and may be optically printed using the so-called negative-positive process where the element is exposed to light through a color negative film which has been processed as described above. The element is sold packaged with instructions to process using a color negative optical printing process, for example, the Kodak RA-4 process, as generally described in PCT WO 87/04534 or U.S. Pat. No. 4,975,357, to form a positive image. Color projection prints may be processed, for example, in accordance with the Kodak ECP-2 process as described in the H-24 Manual. Color print development times are typically 90 seconds or less and desirably 45 or even 30 seconds or less.

Preferred color developing agents are p-phenylenediamines such as:

4-amino-N,N-diethylaniline hydrochloride,

4-amino-3-methyl-N,N-diethylaniline hydrochloride,

4-amino-3-methyl-N-ethyl-N-(2-methanesulfonamidoethyl)aniline sesquisulfate hydrate,

4-amino-3-methyl-N-ethyl-N-(2-hydroxyethyl)aniline sulfate,

4-amino-3-(2-methanesulfonamidoethyl)-N,N-diethylaniline hydrochloride and

4-amino-N-ethyl-N-(2-methoxyethyl)-m-toluidine di-p-toluene sulfonic acid.

Development is usually followed by the conventional steps of bleaching, fixing, or bleach-fixing, to remove silver or silver halide, washing, and drying.

The entire contents of the patents and other publications cited in this specification are incorporated herein by reference. The following examples are intended to illustrate, but not to limit the invention.

### SYNTHETIC EXAMPLE

Dyes of the invention can be prepared in similar manner to Dye-4 which was prepared according to the following scheme:

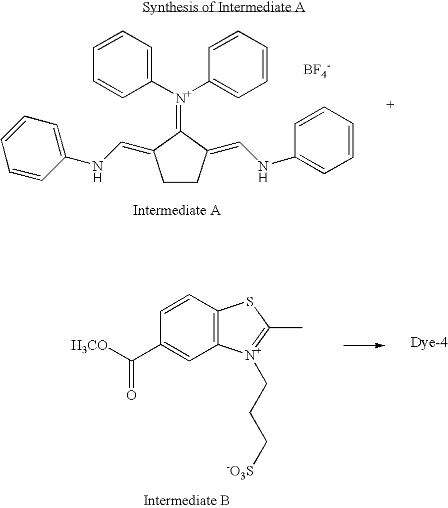

A 12-liter three-necked flask was charged with 1.0 kg diphenylamine, 2.5 liters HOAc, 1.0 kg cyclopentanone and 1.4 kg HBF₄ (48% in water). The mixture was heated to reflux and about 1.4 liter of water/HOAc was distilled off over about 4-5 hrs. The resulting solution (dark blue) was cooled to room temperature, the resulting solid filtered off, which was washed with HOAc, then ether and dried. Yield of enamine: 757 g (40%).

A 5-liter three-necked flask was charged with the above enamine (757 g), 1 kg of ethyl isoformanilide and 1.5 liter butyronitrile. The mixture was heated to reflux and held for an hour (reflux started at 110 C but dropped to 104 C). The heat was turned off and the reaction mixture stirred overnight while allowing to cool to room temperature. The resulting precipitate was filtered off, washed with a little butyronitrile, then with ether. After drying, 996 g (80% yield) of Intermediate A was obtained as a white solid.

### Synthesis of Intermediate B

A 1-liter flask was charged with 272 g sodium sulfide nonahydrate, 39 g sulfur, and about 800 mL MeOH. This was heated to near reflux to dissolve with the addition of a small amount of water to fully dissolve all solids. In a separate 12-liter 3-neck flask was charged 488 g of methyl 3-nitro-4-chlorobenzoate in about 4 liters of McOH. This was warmed to about 55 deg C. and the above sodium sulfide solution added (exothermic) over about 30 minutes with some external cooling. The temperature was kept just below reflux (about 65 deg C.). After about ¼ of the solution added, a yellow precipitate started to form. A few minutes after the addition was complete, the slurry was completely yellow (no brown color). The reaction was heated to reflux and held overnight. The resulting yellow slurry was cooled to room temperature, filtered, washed with MeOH and then with an excess of water. After drying, 437 g (91%) of bis(4-carboxy-2-nitrophenyl)disulfide methyl ester was obtained.

In a 12-liter three-neck flask was charged 437 g of the above disulfide in 2.5 liters HOAc. This mixture (not completely dissolved) was stirred and Zn dust was added in small portions until the temperature went to about 80 deg C. At this point, external cooling with chilled water was started. A total of 700 g Zn was added over about 45 minutes. The reaction mixture became very thick so enough HOAc (about 2.5 liters) was added in portions to maintain stirring. After holding at about 90 deg C. for about 30 minute, 600 mL acetic anhydride was added and held at 80 deg C. for about 1 hour. The reaction thins considerably after a few minutes after the Ac₂O addition but still is a little yellow. After cooling to room temperature, the reaction mixture was filtered through glass fiber and Celite. The resulting solution was stripped to a concentrated oil. As it cooled, the product solidified so about 6 liters of water was added and the cake broken up with stirring to a powder. This was filtered, washed well with water and air-dried.

The above crude product was dissolved in about 4 liters of EtOAc, which was dried over magnesium sulfate, Celite and carbon. After filtering through Celite, the EtOAc was stripped to a thick mush, filtered and washed with a little EtOAc. After drying, 287 g was obtained. A second crop of 50 g was obtained from the mother liquors. These crops were combined and recrystallized from EtOAc to give, after drying, 217 g of 5-methoxycarbonyl-2-methylbenzothiazole.

A 2-liter flask was charged with 217 g of the benzothiazole and 300 mL butyronitrile. This was heated to reflux (dark solution) and filtered into a 2-liter four-neck flask through several layers of glass fiber paper. An additional 50 mL butyronitrile was added. 175 g of propane sultone was added and the reaction heated to reflux (about 124 deg C.) and held for 22 hr. After about 17 hrs the reaction was very thick, so another 100 mL of butyronitrile was added to maintain stirring. After cooling, the resulting benzothiazolium salt (Intermediate B) was filtered off, washed first with butyronitrile, then with ether and finally dried.

### Synthesis of Dye-4

A 4-liter flask was charged with 246 g of Intermediate A and 313 g of Intermediate B. One liter of acetic anhydride was added with stirring followed by 300 ml of triethylamine. The resulting mixture was heated to near reflux over about 20 minutes (about 118 C), held for a few minutes, and cooled to room temperature. The solid was filtered off and washed three times with 300 mL acetic anhydride and then with excess ether. This was returned back to a 4-liter flask which was then charged with about 2 liters MeOH and 100 ml triethylamine with stirring. This was heated to reflux, held a few minutes and allow to cool to room temperature. The solid was filtered off, washed with about 100 mL MeOH, and then with excess ether. After drying, 244 g (52%) of Dye-4 was obtained as fine red crystals.

### Example 1

### Solution Spectra

0.5 g of Dye-1 was dissolved in 5 ml of methanol and its absorbance was measured in solution using a Perkin Elmer UV-Vis spectrophotometer. The wavelength of maximum absorption is reported in Table I as Lₘₐₓ MeOH. 1.0 g of this solution was diluted with 1.0 g of distilled water to allow the dye to form a J-aggregate. The absorbance of this solution was also measured as described above and the wavelength of maximum absorption of the J-aggregate peak is reported in Table I as Lₘₐₓ H₂O.

A solid particle dispersion of Dye-1 was made by adding 2 g of dye and 3 g of a 10% solution of surfactant 10G (Dixie) to 20 g of distilled water. This mixture was added to a 4 oz glass jar along with 60 ml of 1.8 mm zirconium oxide beads and placed on a roller mill at a speed of 70 ft/min for 7 days. After milling, the slurry was passed through a fine metal screen and then rinsed with distilled water to separate the dye slurry from the beads to form a final dispersion of 5% dye.

The solid particle dye dispersion of Dye-1 was coated in a single layer coating format containing 1610 mg/m2 of gelatin on a cellulose acetate support as was identified as Coating 1. Surfactants 10G (Dixie) and Triton X-200E (Rohm & Haas) were employed as coating aids. Coatings of additional dyes were also prepared as described in Table I. All dyes were coated at a level of 50.0 mg/m2. The visible and infrared absorption of each of these coatings was measured from 400 to 1100 nm using a Hitachi U-3410 spectrophotometer and the results are reported in Table I as Lₘₐₓ Film.

TABLE I
Solution Spectra

| | | | | | Lₘₐₓ | | |
|---|---|---|---|---|---|---|---|
| | IR | | | Hammett σ | MeOH | Lₘₐₓ H₂O | Lₘₐₓ Film |
| Ctg # | Dye | Type | Substituent | (R₁ + R₂) | (nm) | (nm) | (nm) |
| 1 | Dye-1 | Inv | Chloro | 0.454 | 809 | 1026 | 1045 |
| 2 | Dye-2 | Inv | Di-chloro | 1.20 | 815 | 966 | 990 |
| 3 | Dye-4 | Inv | COOMe | 0.78 | 804 | 966 | 980 |
| 4 | Dye-5 | Inv | CN | 1.32 | 802 | 981 | 980 |
| 5 | Dye-6 | Inv | CF₃ | 1.08 | 802 | 981 | 1015 |
| 6 | Dye-7 | Inv | NO₂ | 1.56 | 823 | 1006 | 950 |
| 7 | C-1 | Comp | Di-methoxy | −0.306 | 863 | 737/1078 | 770/1075 |

These results clearly demonstrate that the dyes of the present invention form J-aggregate peaks at the desired longer wavelengths when diluted with water and when coated in an aqueous gelatin film layer as evidenced by the large spectral shift compared to their peak absorption when dissolved in methanol. However, comparison dye C-1 that did not meet the Hammett σ parameters, showed much shorter peak absorptions in water (737 nm) and in film (770 nm) and exhibited only weak J-aggregate peaks in water (1078 nm) and in film (1075 nm).

### Example 2

### Dispersion Spectra

Liquid Crystalline Suspension (LCS) Preparation: 1.0 g of Dye-4, 0.2 g of 5.0% solution of Promexal X50 biocide (Zeneca), and 0.02 g of Lumulse 42-OK antifoamant (Lambent) was added to 98.78 g of distilled water in a 250 ml glass beaker and was stirred with a 4 cm Cowels mixer at 1100 rpm for 15 min followed by a Silverson mixer at 5000 rpm for 30 min at room temperature. A microscopic examination using crossed polarizing filters at 100× magnification revealed a coarse, grainy appearance with large domains of birefringence indicating that the dye was present in the liquid crystalline state. No large crystals of dye greater then 1 micron were evident. 1.0 g of comparison dye C-2 was subjected to the same procedure described above. In this case, microscopic examination showed no indication of liquid crystals and the mixture was loaded with many large (10-50 microns) crystals of dye.
Solid Particle Dispersion (SPD) Preparation: 1.0 g of Dye-4 and 1.5 g of a 10% solution of surfactant 10G (Dixie) and 97.5 g of distilled water was added to a 16 oz glass jar containing 845.0 g of 1.8 mm zirconium oxide beads and was placed on a roller mill at a speed of 97 rpm for 4 days. After milling, the slurry was passed through a fine metal screen to separate the dye slurry from the beads to form a final dispersion of 1.0% dye. A microscopic examination using crossed polarizing filters at 100× magnification showed that the dispersion had a coarse, grainy birefringence with many small particles and no dye particles greater than 1 micron. 1.0 g of comparison dye C-2 was subjected to the same procedure described above. In this case, microscopic examination showed very weak birefringence and many large (5-20 micron) particles.

These dispersions and suspensions were coated in Layer 1 of the following bi-layer coating format as described below (coated levels are given in mg/m2):
Support: Cellulose acetate
Layer 1: Gelatin (2010), IR dye (20)
Layer 2: Gelatin (828), PDMS (polydimethylsiloxane) lubricant (39), Polymeric matte beads (5.3)

The spectral absorbance of these raw stock coatings was measured from 350 to 1100 nm using a Hitachi U-3410 spectrophotometer. The wavelength of maximum absorption and density at that wavelength are given in Table II along with their Status M red, green, and blue densities.

TABLE II
Dispersion Spectra

| Ctg | IR | | Disper- | Lₘₐₓ | Density | | | |
|---|---|---|---|---|---|---|---|---|
| No | Dye | Type | sion | (nm) | @Lₘₐₓ | Red | Green | Blue |
| 8 | None | Comp | — | — | 0.06 | 0.07 | 0.07 | 0.06 |
| 9 | Dye- | Inv | LCS | 980 | 1.18 | 0.08 | 0.08 | 0.06 |
| | 4 | | | | | | | |
| 10 | Dye- | Inv | SPD | 980 | 0.71 | 0.08 | 0.08 | 0.06 |
| | 4 | | | | | | | |
| 11 | C-2 | Comp | LCS | 980 | 0.09 | 0.08 | 0.08 | 0.06 |
| 12 | C-2 | Comp | SPD | 980 | 0.08 | 0.09 | 0.09 | 0.07 |
| 13 | None | Comp | — | — | 0.06 | 0.07 | 0.07 | 0.06 |

These data clearly show that the dye of the present invention provides a much higher absorption at 980 nm relative to the comparison dye, which meets the Hammett σ parameters limitations but which lacks solubilization on the alkyl substituents on the nitrogens. The results also illustrate that the liquid crystalline suspension (LCS) process is a preferred method of incorporation into film coatings to maximize the infrared absorbance.

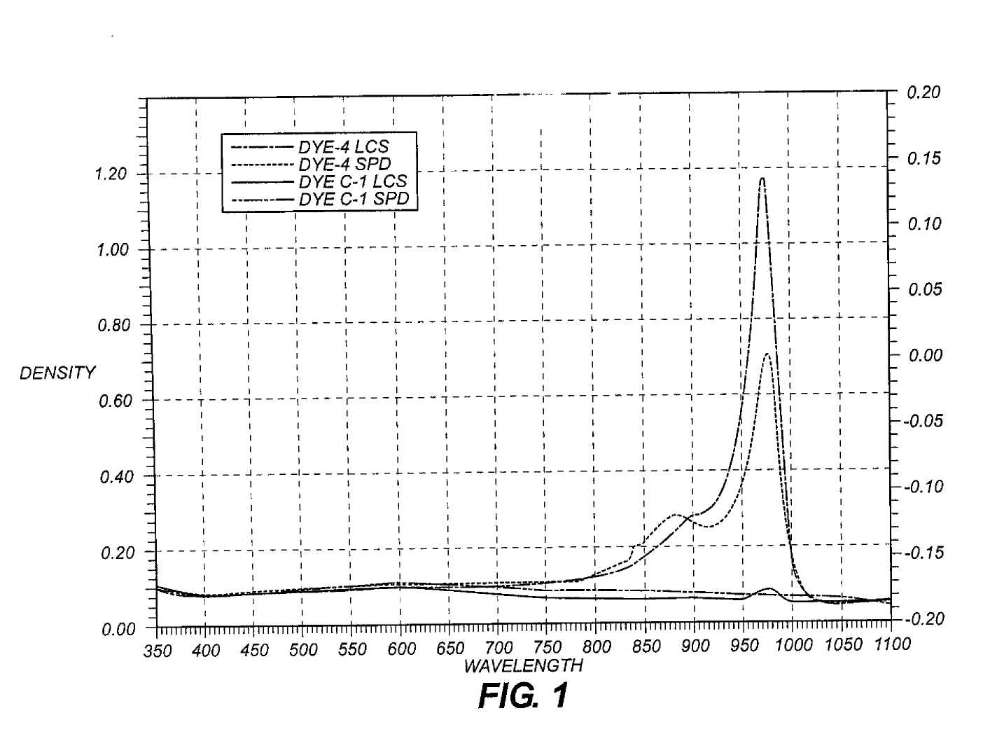

FIG. 1 shows the absorbance of coating numbers 9-12. Dye-4 clearly shows the formation of a J-aggregate at about 980 nm in both of these dispersion formulations. A liquid-crystalline dispersion gives more IR density at Lₘₐₓ compared to a solid particle dispersion. Comparative IR dye C-1 does not form significant amounts of J-aggregate under either of these dispersion conditions and so has low density at 980 nm. Dye-4 also has very little unwanted visible absorption under these conditions.

The structures of the comparative dyes in Examples 1 and 2 were:

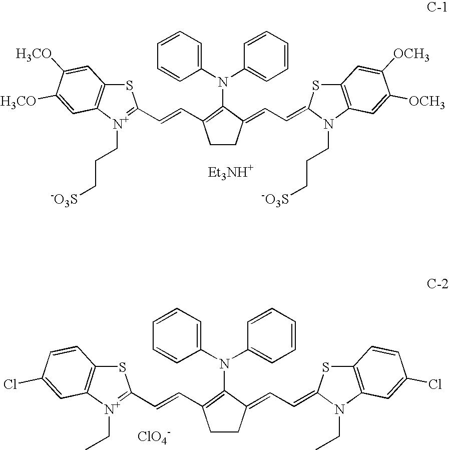

### Example 3

### IR Dyes in Multilayer Photographic Format

Multilayer films demonstrating the principles of this invention were produced by coating the following layers on a cellulose triacetate film support (coverage are in grams per meter squared, emulsion sizes as determined by the disc centrifuge method and are reported in diameter×thickness in micrometers). Surfactants, coating aids, emulsion addenda (including 4-hydroxy-6-methyl-1,3,3a,7-tetraazaindene), sequestrants, thickeners, lubricants and tinting dyes were added to the appropriate layers as is common in the art. Couplers and other non-water soluble materials were added as conventional oil-in-water dispersions as known in the art

### Example ML-1

Layer 1 (Antihalation layer): gelatin at 2.01, colloidal metallic silver at 0.320; ILS-1 at 0.160; DYE-1 at 0.053; DYE-2 at 0.090; Potassium iodide at 0.007 and a mixture of UV-1 and UV-2 at 0.083 each
Layer 2 (Slow cyan layer): a blend of two red-sensitized tabular silver iodobromide emulsions: (i) a 0.72×0.11, 4.5% I (sensitized with a mixture of RSD-2 and RSD-3) at 0.040, (ii) a 0.55×0.08, 1.5% I (sensitized with a mixture of RSD-1 and RSD-2) at 0.152; cyan dye-forming couplers C-1 at 0.170, C-2 at 0.056 and C-3 at 0.095; bleach accelerator releasing coupler B-1 at 0.073; image modifier D-1 at 0.008; D-2 at 0.024; masking coupler MC-1 at 0.020 and gelatin at 1.50.
Layer 3 (Mid cyan layer): a blend of two red-sensitized (both with a mixture of RSD-2 and RSD-3) iodobromide tabular emulsions: (i) a 1.25×0.12, 3.7% I at 0.045 and (ii) a 0.72×0.11 micron, 4.5 mole % I at 0.163; C-1 at 0.100; C-2 at 0.033; Y-1 at 0.090; B-1 at 0.017; D-1 at 0.040; D-2 at 0.019; MC-1 at 0.018; B-1 at 0.017 and gelatin at 0.82.
Layer 4 (Fast cyan layer): a blend of two red-sensitized (both with a mixture of RSD-2 and RSD-3) iodobromide tabular emulsions: (i) 2.0×0.13 microns, 3.7 mole % I at 0.060 and (ii) 1.25×0.12 micron, 3.7 mole % I at 0.180; C-1 at 0.023; C-3 at 0.020; D-2 at 0.013; B-1 at 0.010; MC-1 at 0.019 and gelatin at 0.45.
Layer 5 (Interlayer): ILS-1 at 0.066; S-1 at 0.003 and gelatin at 0.446.
Layer 6 (Slow magenta layer): a blend of two green sensitized (both with a mixture of GSD-1 and GSD-2) emulsions: (i) 0.36×0.13 micron, 4.8 mole % iodide at 0.045 and (ii) 0.55×0.08, 1.5 mole % iodide at 0.081; magenta dye-forming coupler M-1 at 0.154; MC-2 at 0.125; yellow image modifier D-3 at 0.024 and gelatin at 1.063.
Layer 7 (Mid magenta layer): a blend of two green-sensitized (both with a mixture of GSD-1 and GSD-2) silver iodobromide tabular emulsions: (i) 0.36×0.13 microns, 4.8 mole % iodide at 0.180 and (ii) 0.78×0.11 microns, 4.5 mole % iodide at 0.130; M-1 at 0.062; MC-2 at 0.050; D-3 at 0.010; D-1 at 0.010; ILS-2 at 0.011 and gelatin at 0.981.
Layer 8 (Fast magenta layer): a blend of two green-sensitized silver iodobromide tabular emulsions: (i) 1.27×0.13 micron, 6 mole % iodide (sensitized with a mixture of GSD-1, GSD-2 and GSD-3) at 0.100 and (ii) 0.78×0.11 microns, 4.5 mole % iodide (sensitized with a mixture of GSD-1 and GSD-2 at 0.050; addenda H-1 at 0.010; M-1 at 0.030; MC-2 at 0.033 and gelatin at 1.063.
Layer 9 (Interlayer): ILS-1 at 0.072, S-1 at 0.040 and gelatin at 0.490.
Layer 10 (Slow yellow layer): A blend of three blue sensitized emulsions: (i) 1.60×0.13 micron, 3 mole % iodide (sensitized with BSD-1) at 0.055, (ii) 0.75×0.13 microns, 3 mole % iodide (sensitized with a mixture of BSD-1 and BSD-2) at 0.145 and (iii) 0.38×0.12 microns, 3 mole % iodide (sensitized with a mixture of BSD-1 and BSD-2) at 0.210; Y-1 at 0.900; D-6 at 0.033; D-1 at 0.016; B-1 at 0.010 and gelatin at 1.611 with bis(vinylsulfonyl)methane hardener at 1.8% of total gelatin weight is streamed into this layer during application to the support.
Layer 11 (Fast yellow layer): A blend of two blue sensitized emulsions: (i) 2.8×0.12 microns, 4.2 mole % iodide (sensitized with a mixture of BSD-1 and BSD-2) at 0.220 and (ii) 1.60×0.13 microns, 3 mole % iodide (sensitized with BSD-1) at 0.115; Y-1 at 0.245; D-6 at 0.088; B-1 at 0.005 and gelatin at 0.650.
Layer 12 (UV Filter Layer): silver bromide Lippman emulsion at 0.210; UV-1 and UV-2 both at 0.115 and gelatin at 0.560.
Layer 13 (Protective overcoat): a blend of permanent and soluble Matte beads and gelatin at 0.867.

Formulas for materials used in the above formats are as follows:

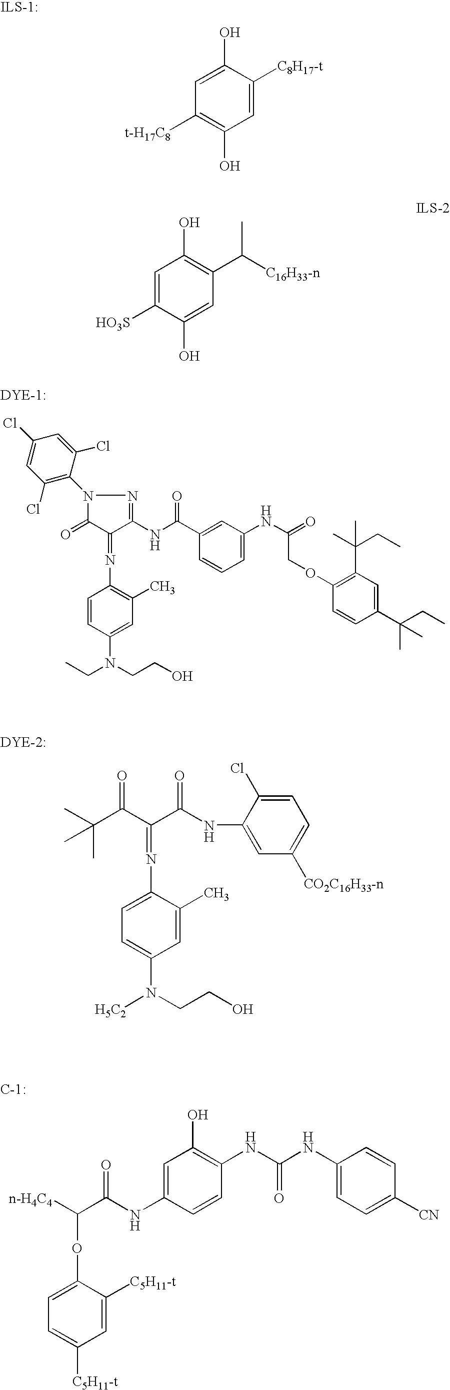

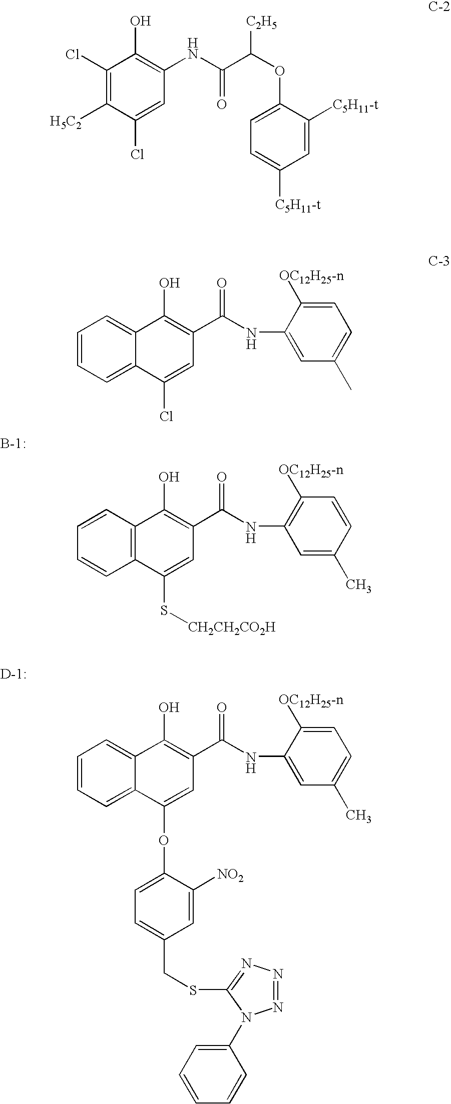

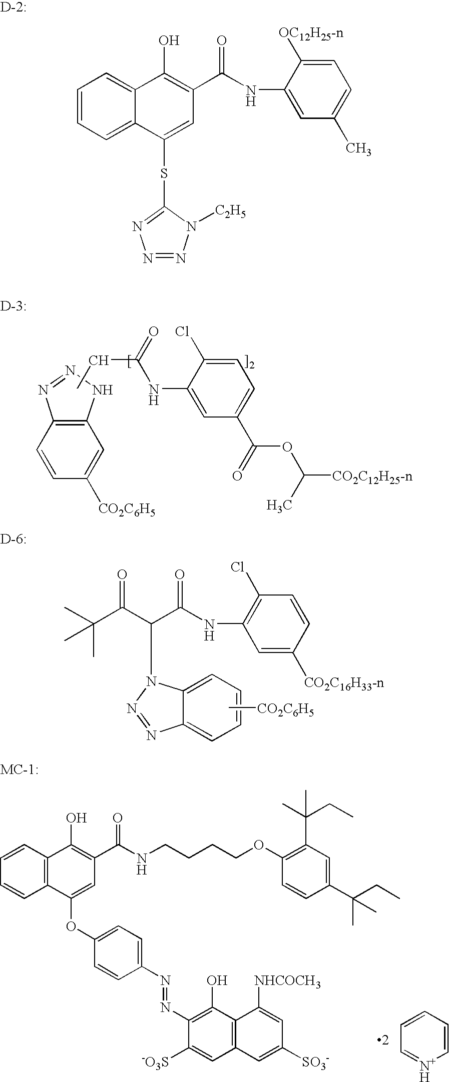

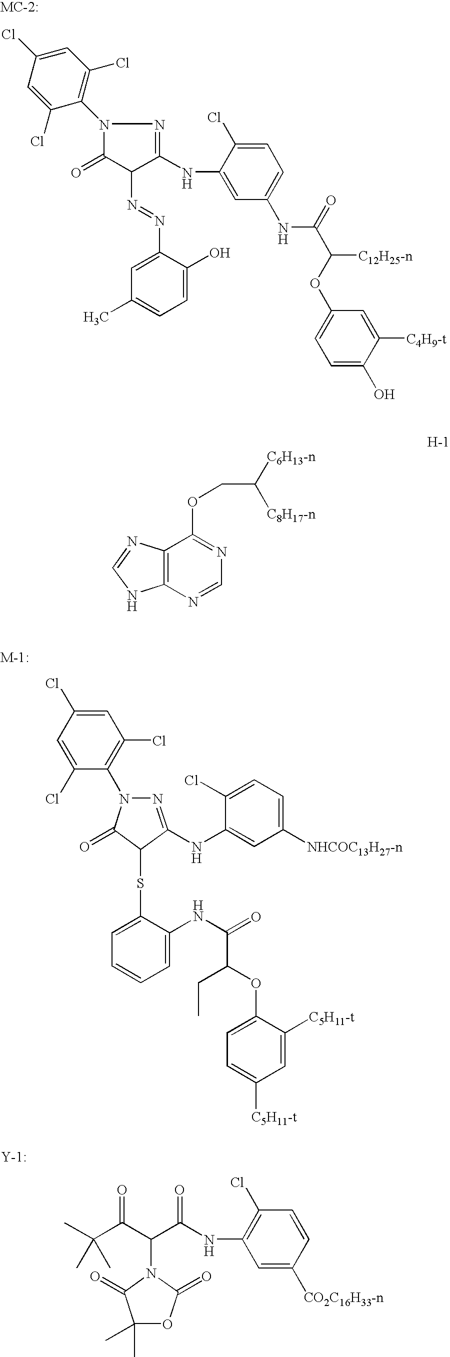

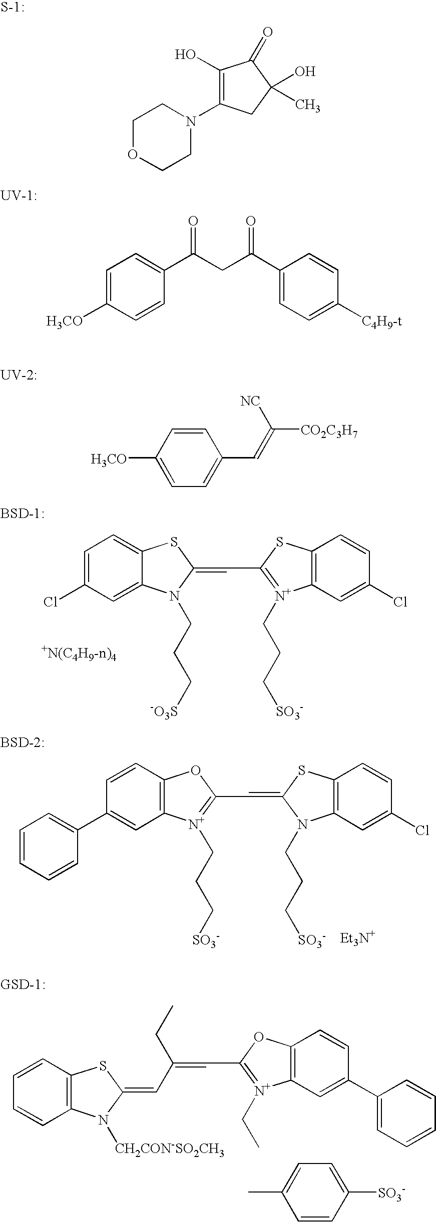

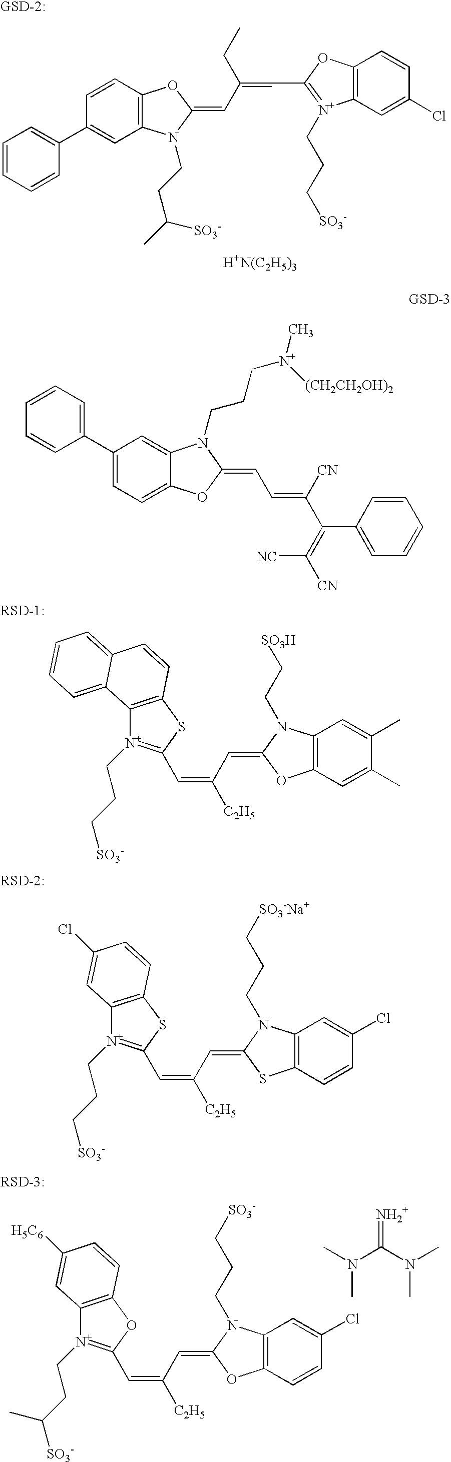

Samples ML-2 to ML-5 (total silver present=2.381 g/m²) were prepared as ML-1 (total silver present=2.501 g/m²) except for the changes indicated in Table 1. Samples ML-6 to ML-9 was prepared as ML-4 except for the changes in location of the IR dye (at 18 mg/m²) as indicated in Table 2.

The above multilayer coatings were given a neutral stepped exposure, followed by processing in the KODAK FLEXICOLOR™ (C-41) process as described in British Journal of Photography Annual, 1988, pp 196-198. The red density at Dmin (fog) was measured as well as the IR density at 950 nm. Results for samples ML-1 to ML-5 are shown in Table 1. The data is relative to ML-1=1.0.

TABLE 1
Effect of lower colloidal silver and Dye-1 on Red Fog

| | Colloidal Ag | Dye-1 in | | |
|---|---|---|---|---|
| | in Layer 1 | Layer 1 | Normalized | Normalized |
| Sample | (g/m2) | (mg/m2) | Fog | IR Density |
| ML-1 (Comp) | 0.320 | 0 | 1.0 | 1.0 |
| ML-2 (Comp) | 0.200 | 0 | 0.9 | 0.74 |
| ML-3 (Inv) | 0.200 | 10 | 0.7 | 0.85 |
| ML-4 (Inv) | 0.200 | 18 | 0.7 | 0.97 |
| ML-5 (Inv) | 0.200 | 26 | 0.7 | 1.09 |

It can be seen in the table above that red fog can be improved by reduction of colloidal silver (ML-1 versus ML-2). However the IR density at 950 nm is also severely reduced. The introduction of IR dye (ML-3 to ML-5) not only compensates for the IR loss allowing the reduction of colloidal silver with the concomitant fog reduction but surprisingly lowers the fog even further. This allows for low silver films with equivalent IR density but with an improved fog position.

The location of the IR dye can be important to obtain the fog reduction. Results for ML-6 to ML-9 are shown in Table 2. The data is relative to ML-1=1.0.

TABLE 2
Placement of IR dye

| | Colloidal Ag | | | |
|---|---|---|---|---|
| | in Layer 1 | Location of | Normalized | Normalized |
| Sample | (g/m2) | Dye-1 | Fog | IR Density |
| ML-1 (Comp) | 0.320 | None | 1.0 | 1.0 |
| ML-2 (Comp) | 0.200 | None | 0.9 | 0.74 |
| ML-4 (Inv) | 0.200 | Layer 1 | 0.7 | 0.97 |
| | | (AHU) | | |
| ML-6 (Comp) | 0.200 | Layer 4 (FC) | 1.4 | 1.0 |
| ML-7 (Inv) | 0.200 | Layer 5 (IL) | 0.9 | 1.04 |
| ML-8 (Inv) | 0.200 | Layer 9 (IL) | 0.9 | 1.04 |
| ML-9 (Inv) | 0.200 | Layer 12 | 0.9 | 1.02 |
| | | (UV) | | |

It can readily be seen in Table 2 that increases in IR density can be achieved by any location of the IR dye. While the fog suppression is best achieved when the material is placed in layer 1 the AHU layer. Placement in a non-imaging layer (for example, an interlayer as in ML-7 and ML-8 or the UV layer as in ML-9) does not impact fog levels. However, location in an imaging layer (the fast cyan as in ML-6) increases fog.

The invention has been described in detail with particular reference to certain preferred embodiments thereof, but it will be understood that variations and modifications can be effected within the spirit and scope of the invention.

1.-11. (canceled)

12. An infrared dye having the structure according to Formula (III): 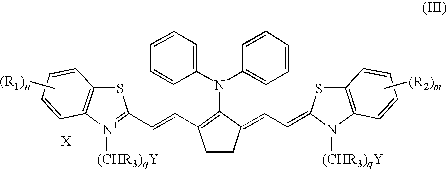
wherein:
R₁ and R₂ are the same and each is a halide, an ester of a carboxylic acid (—CO₂R), a carbamoyl group (—CONR), a sulfoxide, group, a sulfone group, a cyano group, a heterocyclic group or a nitro group and chosen such that the sum of the Hammett σₚₐᵣₐ and σₘₑₜₐ parameters of R₁ and R₂ is greater than 0.75;
n and m each independently represents an integer 1 or 2;
R₃ is hydrogen or an alkyl group;
q is 3 or 4; and
Y is a sulfonic acid group; and
X is a counterion chosen so that the net charge of the entire molecule is zero.

13. The infrared dye of claim 12 as a solid particle or a liquid-crystalline dispersion with a maximum absorption between 900 nm and 1010 nm

14. The solid particle or a liquid-crystalline dispersion of claim 13 wherein n is 1 and R₁ and R₂ are each is an ester of a carboxylic acid (—CO₂R).

15. The solid particle or a liquid-crystalline dispersion of claim 14 where the maximum absorption is between 920 nm and 980 nm.

16. The dispersion of claim 15 which is a liquid-crystalline dispersion.

17. The infrared dye of claim 12 chosen from: 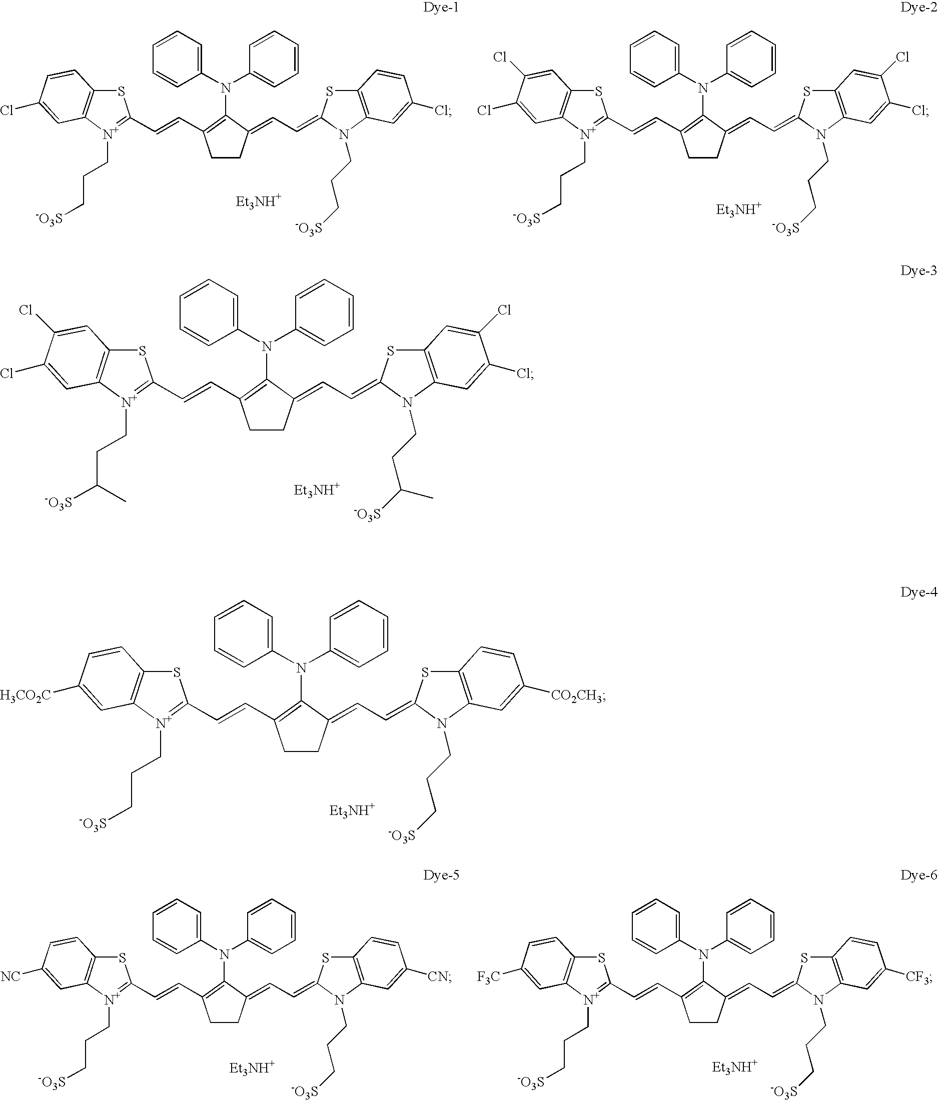 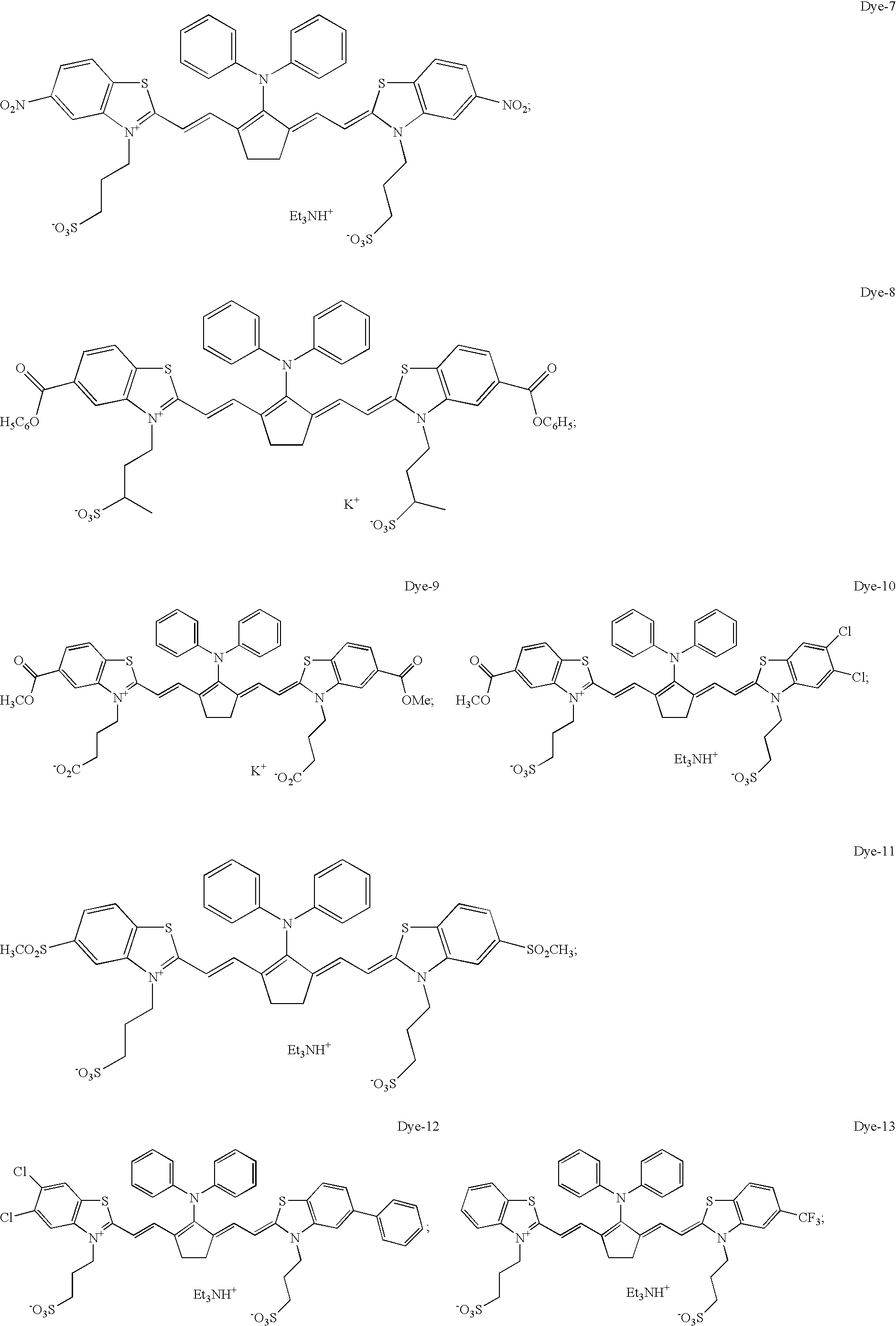 [chemical structure]
